(19) United States
(12) Patent Application Publication
(10) Pub. No.: US 2023/0292765 A1
(43) Pub. Date: Sep. 21, 2023

(54) NEW AGROCHEMICAL FORMULATIONS FOR FUSARICIDIN PRODUCING BACTERIA

(71) Applicant: BASF SE, Ludwigshafen (DE)

(72) Inventors: Ulrike Malang, Limburgerhof (DE); Ralph Steinbacher, Limburgerhof (DE); Christina Arends, Limburgerhof (DE); Matthias Bratz, Limburgerhof (DE)

(21) Appl. No.: 18/018,383

(22) PCT Filed: Jul. 20, 2021
(86) PCT No.: PCT/EP2021/070198

§ 371 Date: Jan. 27, 2023

(30) Foreign Application Priority Data
Jul. 31, 2020 (EP) 20188892.2
May 17, 2021 (EP) 21174006.3

(51) Int. Cl. A01N 63/25; C12N 1/20; A01N 25/04; A01P 3/00; A01N 25/30; A01N 25/22

(52) U.S. Cl. CPC A01N 63/25; C12N 1/20; A01N 25/04; A01P 3/00; A01N 25/30; A01N 25/22; C12R 2001/12

(57) ABSTRACT

The present invention is directed to spraying liquid suitable for spraying plants, comprising spores of fusaricidin producing bacteria, at least one category (1) additive and/or at least one category (2) additive and less than 81 g per hectare of a category (3) additive, to solid and liquid formulations comprising spores of fusaricidin producing bacteria, at least one category (1) additive and/or at least one category (2) additive and optionally a category (3) additive, as well as to methods to control phytopathogenic fungi on plants applying such spraying liquids or formulations.

The present invention is directed to spraying liquids suitable for spraying plants, comprising spores of fusaricidin producing bacteria, at least one category 1 additive and/or at least one category 2 additive and less than 81 g per hectare of a category 3 additive. The present invention is further directed to solid and liquid formulations comprising spores of fusaricidin producing bacteria, at least one category 1 additive and/or at least one category 2 additive and optionally a category 3 additive, as well as to methods to control phytopathogenic fungi on plants by applying such spraying liquids or formulations to the plants.

### FIELD OF THE INVENTION

In its main aspects the invention refers to:

Spraying liquids suitable for spraying plants, comprising
a. biomass comprising spores of fusaricidin producing bacteria, in an amount of 50 g per hectare to 2000 g per hectare,
b. at least one category 1 additive or at least one category 2 additive or a mixture of at least one category 1 additive and at least one category 2 additive, in a total amount of 20 g per hectare to 2000 g per hectare, and
c. less than 81 g per hectare of category 3 additives,
   wherein the at least one category 1 additive is selected from at least one group of additives 1a) to 1f):
1a) poloxamers, polyethylene glycol (A)—polypropylene glycol (B) block copolymers of the A-B-A or B-A-B type, comprising polyethylene glycol-polypropylene glycol-polyethylene glycol block polymers with a molar mass of approximately 950 to 6500 g/mol and a polyethylene oxide content of 10% to 50% w/w.;
1b) polyvinylpyrolidines, polyvinylacetates, and statistical and block copolymers of polyvinyl pyrrolidone and polyvinyl acetate, all with a molar mass of approximately 9000 to 70000 g/mol;
1c) polyoxyethylene sorbitol fatty acid esters (“polysorbates”), having a fatty acid chain with a length of 10 to 20 carbon atoms, preferably 12 to 18 carbon atoms, most preferably dodecanoic acid and oleic acid and linoleic acid, from 10 to 150 oxyethylene units, preferably 20 to 80 oxyethylene units, most preferred 20 to 40 oxyethylene units;
1d) fatty acid glycerol esters including fatty acid mono- and diesters of glycerol and polyglycerol, glycerolmonooleate, glyceroldioleate;
1e) alcohol alcoxylates, preferably C10-18 alcohols, more preferably C10-C13 alcohols, most preferably C10 alcohols, from 5 to 80 oxyethylene units, more preferably from 5 to 40 oxyethylene units, most preferred from 7 to 14 oxyethylene units;
1f) alkyl glucosides having an alkyl chain length C5-C15 and 1 to 10 glucoside units, and
   wherein the at least one category 2 additive is selected from at least one group of additives 2a) to 2c)
2a) polyethylene glycol-grafted polyacrylates, and salts thereof, preferably sodium salts thereof;
2b) ethoxylated polyethylene imines;
2c) sodium lauryl ether sulfates, sodium dodecylpoly(oxyethylene)sulfates with 1 to 50 oxyethylene units, preferably with 1 to 7 oxyethylene units, most preferred with 1 to 4 oxyethylene units,
   wherein the category 3 additive belongs to a group of additives 3a) to 3g)
3a) lignosulfonates;
3b) alkylbenzene sulfonates and salts thereof;
3c) alkylnaphthalene sulfonates and salts thereof;
3e) napthalene sulfonic acid co-condensates;
3f) alkyl sulfonates and salts thereof;
3g) fatty alcohol sulfates and salts thereof, preferably sodium lauryl sulfate.

Preferably the spraying liquids comprise, in an order of preference, less than 75 g, 70 g, 65 g, 60 g, 55 g, 50 g, 45 g, 40 g, 35 g, 30 g, 25 g, 20 g, 15 g, 10 g, 5 g, 4 g, 3 g, 2 g, or less than 1 g per hectare of the total of category 3 additives.

In one embodiment they comprise no category 3 additive.

Further aspects are kits of at least two parts to prepare such spraying liquids.

The invention includes also solid or liquid formulations, preferably a wettable powder (WC) or suspension concentrate (SC) formulation, comprising:
a. 5% w/w to 50% w/w biomass comprising spores of fusaricidin producing bacteria,
b. 5% w/w to 50% w/w of at least one category 1 additive or at least one category 2 additive or a mixture of at least one category 1 additive or at least one category 2 additive, and
c. less than 2% w/w of category 3 additives,
   wherein the at least one category 1 additive is selected from at least one group of additives 1a) to 1f):
1a) poloxamers, polyethylene glycol, polypropylene glycol or polyethylene glycol block polymers with a molar mass of approximately 950 to 6500 g/mol and a polyethylene oxide content of 10% to 50% w/w;
1b) polyvinylpyrolidines, polyvinylacetates and statistical and block copolymers of polyvinyl pyrrolidone and polyvinyl acetate, all with a molar mass of approximately 9000 to 70000 g/mol;
1c) polyoxyethylene sorbitol fatty acid esters (“polysorbates”), having a fatty acid chain with a length of 10 to 20 carbon atoms, preferably 12 to 18 carbon atoms, most preferably dodecanoic acid and oleic acid and linoleic acid, from 10 to 150 oxyethylene units, preferably 20 to 80 oxyethylene units, most preferred 20 to 40 oxyethylene units;
1d) fatty acid glycerol esters, fatty acid mono- and diesters of glycerol and polyglycerol, glycerolmonooleate, glyceroldioleate, polyglycerol fatty acid ester;
1e) alcohol alcoxylates, preferably C10-18 alcohols, more preferably C10-C13 alcohols, most preferably C10 alcohols, from 5 to 80 oxyethylene units, more preferably from 5 to 40 oxyethylene units, most preferred from 7 to 14 oxyethylene units;
1f) alkyl glucosides having an alkyl chain length C5-C15 and 1 to 10 glucoside units,
and the at least one category 2 additive is selected from at least one group of additives 2a) to 2c)
2a) polyethylene glycol-grafted polyacrylates, and salts thereof, preferably sodium salts thereof;
2b) ethoxylated polyethyleneimines;
2c) sodium lauryl ether sulfate, preferably sodium dodecylpoly(oxyethylene)sulfates with 1 to 50 oxyethylene units, preferably with 1 to 7 oxyethylene units, most preferred with 1 to 4 oxyethylene units,
and the category 3 additives are selected from the groups of additives 3a) to 3g)
3a) lignosulfonates;
3b) alkylbenzene sulfonates and salts thereof;
3c) alkylnaphthalene sulfonates and salts thereof;
3e) apthalene sulfonic acid co-condensates;
3f) alkyl sulfonates and salts thereof;
3g) fatty alcohol sulfates and salts thereof, preferably sodium lauryl sulfate.

The invention further refers to a solid or liquid formulation, preferably a wettable powder (WP) or suspension concentrate (SC) formulation according to any one of claims 19 to 29 comprising
a. 5.0% w/w to 50% w/w biomass comprising spores of fusaricidin producing bacteria,
b. 2.0% w/w to 50% w/w of a category 1 additive,
c. 0% w/w to 50% w/w of a category 2 additive,
d. 0 w/w to 2% w/w of a category 3 additive, and
e. 20% w/w to 75% w/w of auxiliaries.
   wherein the total amounts of a, b, c, d, and e are selected to result in a total amount of 100% w/w.

The invention also refers to a method to control phytopathogenic fungi on plants comprising
a. providing at least one solid or liquid formulation, preferably a wettable powder (WP) or suspension concentrate (SC) formulation, as described above in a total amount of 0.5 kg to 4 kg per hectare or a kit of parts as described above,
b. adding water to a final amount of 100 liters to 1000 liters per hectare, and
c. spraying the resulting spraying liquid on plants.

Further aspects are processes for making solid formulation, preferably a wettable powder (WP) formulation, comprising:
a. 5.0% w/w to 50% w/w biomass comprising spores of fusaricidin producing bacteria,
b. 5.0% w/w to 50% w/w of a category 1 additive,
c. 0% w/w to 20% w/w of a category 2 additive,
d. 0 w/w to 2% w/w of a category 3 additive, and
e. 30% w/w to 50% w/w of auxiliaries.
   wherein the total amounts of a, b, c, d, and e are selected to result in a total amount of 100% w/w, comprising the following steps:
A. providing biomass comprising spores of fusaricidin producing bacteria,
B. adding at least one category 1 additive,
C. optionally adding at least one category 2 additive,
D. optionally adding at least one category 3 additive,
E. optionally adding auxiliaries,
F. drying the biomass containing slurry by spray-drying, vacuum-drum drying, fluidized-bed drying or spray-granulation,
Wherein steps B), C), D), and E) can be carried out at any time during the process.

Also included are processes for making a suspension concentrate (SC) formulation comprising:
a. providing biomass comprising spores of fusaricidin producing bacteria,
b. dispersing said biomass by shear or mechanical energy input, preferably in a rotor-stator colloid mill or a bead mill in a continuous phase comprising,
c. adding at least one category 1 additive,
d. optionally adding at least one category 2 additive,
e. optionally adding at least one category 3 additive,
f. optionally adding auxiliaries,
   wherein steps c), d), e) and f) can be carried out at any time during the process.

### BACKGROUND OF THE INVENTION

Biocontrol agents, also referred to as “microbials” or “biologicals” play an increasingly important role for protecting crops against various pests. In particular Bacillus species are well known for fungicidal, insecticidal and nematicidal activity. Such biologicals are often applied as aqueous or non-aqueous formulations of the respective spores. For controlling phytopathogenic fungi several microbial pesticides comprising spore-forming bacteria, including Paenibacillus species, have been described.

WO1999/059412 discloses a Paenibacillus polymyxa strain PKB1 (bearing ATCC accession no. 202127) active against several phytopathogenic fungi.

WO2006/016558 discloses Paenibacillus sp. strains BS-0048, BS-0074, BS-0277 and P. polymyxa strain BS-0105 as well as fusaricidin A and fusaricidin B for protection of plants from infections with fungi. A further antifungal Paenibacillus strain BRF-1 has been isolated from soybean rhizosphere (African J. Microbiol. Res. 4(24), 2692-2698, 2010).

WO 2011/069227 discloses a P. polymyxa strain JB05-01-1 (bearing ATCC accession no. PTA-10436) having a highly inhibitory effect against pathogenic bacteria, pre-dominantly food-borne human pathogenic bacteria.

Budi et al. (Appl Environ Microbiol, 1999, 65, 5148-5150) have isolated Paenibacillus sp. strain B2 from mycorrhizosphere of Sorghum bicolor having antagonistic activity towards soil borne fungal pathogens like Phytophthora parasitica.

The antimicrobial activity of numerous Paenibacillus strains, i. a. a P. peoriae strain, against numerous bacterial, fungal and yeast pathogens has been reported (Lett. Appl. Microbiol. 43, 541-547, 2006).

Raza et al. (Brazilian Arch. Biol. Techol. 53, 1145-1154, 2010; Eur. J. Plant Pathol. 125: 471-483, 2009) described a fusaricidin-type compound-producing Paenibacillus polymyxa strain SQR-21 effective against Fusarium oxysporum.

Fusaricidins are a group of cyclic lipodepsipeptide antibiotics, which can be isolated from Paeni bacillus spp., Their common structural features are: a macrocyclic ring consisting of six amino acid residues, three of which are L-Thr, D-allo-Thr and D-Ala, as well as the 15-guanidino-3-hydroxypentadecanoic acid tail attached to the N-terminal L-Thr residue by an amide bond (ChemMedChem 7, 871-882, 2012; J. Microbiol. Meth. 85, 175-182, 2011, Table 1 herein).

These compounds are cyclized by a lactone bridge between the N-terminal L-Thr hydroxyl group and the C-terminal D-Ala carbonyl group. The position of the amino acid residues within the depsipeptide cycle are usually numbered starting with the abovementioned L-Thr which itself also carries the GHPD chain and ending with the C-terminal D-Ala. Non-limiting examples of fusaricidins isolated from Paenibacillus are designated LI-F03, LI-F04, LI-F05, LI-F07 and LI-F08 (J. Antibiotics 40(11), 1506-1514, 1987; Heterocycles 53(7), 1533-1549, 2000; Peptides 32, 1917-1923, 2011) and fusaricidins A (also called LI-F04a), B (also called LI-F04b), C (also called LI-F03a) and D (also called LI-F03b) (J. Antibiotics 49(2), 129-135, 1996; J. Antibiotics 50(3), 220-228, 1997). The amino acid chain of a fusaricidin is not ribosomally generated but is generated by a non-ribosomal peptide synthetase. Structural formulae of known fusaricidins are shown in Table 1 (Biotechnol Lett. 34, 1327-1334, 2012; FIG. 1 therein). The compounds designated as LI-F03a, LI-F03b up to LI-F08a and LI-F08b are herein also referred to as fusaricidins LI-F03a, LI-F03b up to LI-F08a and LI-F08b due to their structure within the fusaricidin family (see Table 1).

TABLE 1
Structures of the fusaricidin family.

| Fusaricidin | X² | X³ | X⁵ |
|---|---|---|---|
| A (LI-F04a) | D-Val | L-Val | D-Asn |
| B (LI-F04b) | D-Val | L-Val | D-Gln |
| C (LI-F03a) | D-Val | L-Tyr | D-Asn |
| D (LI-F03b) | D-Val | L-Tyr | D-Gln |
| LI-F05a | D-Val | L-Ile | D-Asn |
| LI-F05b | D-Val | L-Ile | D-Gln |
| LI-F06a | D-allo-Ile | L-Val | D-Asn |
| LI-F06b | D-allo-Ile | L-Val | D-Gln |
| LI-F07a | D-Val | L-Phe | D-Asn |
| LI-F07b | D-Val | L-Phe | D-Gln |
| LI-F08a | D-Ile | L-allo-Ile | D-Asn |
| LI-F08b | D-Ile | L-allo-Ile | D-Gln |

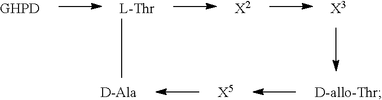

wherein an arrow defines a single (amide) bond either between the carbonyl moiety of GHPD and the amino group of L-Thr (L-threonine) or between the carbonyl group of one amino acid and the amino group of a neighboring amino acid, wherein the tip of the arrow indicates the attachment to the amino group of said amino acid L-Thr or of said neighboring amino acid; and wherein the single line (without an arrow head) defines a single (ester) bond between the carbonyl group of D-Ala (D-alanine) and the hydroxyl group of L-Thr; and wherein GHPD is 15-guanidino-3-hydroxypentadecanoic acid. Further fusaricidin-like compounds known in the art include Paeniserine A1, Paeniserine A2, Paeniserine A3, Paeniserine A4, Paeniserine BI, Paeniserine B2, Paeniserine B3, Paeniserine B4, Paeniserine CI, Paeniserine C2, Paeniserine C3, Paeniserine C4, Paeniprolixin A 1, Paeniprolixin A2, Paeniprolixin B1, Paeniprolixin B2, Paeniprolixin CI, Paeniprolixin C2, Paeniprolixin DI, Paeniprolixin D2, Paeniprolixin E1, Paeniprolixin E2, Paeniprolixin FI, Paeniprolixin F2, as for example described in WO16154297.

WO 2007/086645 describes the fusaricidin synthetase enzyme and its encoding gene as isolated from Paenibacillus polymyxa strain E681 which enzyme is involved in the synthesis of fusaricidins A, B, C, D, LI-F03, LI-F04, LI-F05, LI-F07 and LI-F08.

The genome of several Paenibacillus polymyxa strains has been published so far: inter alia for
P. polymyxa ATCC 842 T in Jeong H, Park S Y, Chung W H, Kim S H, Kim N, Park S H, et al. Draft genome sequence of the Paenibacillus polymyxa type strain (ATCC 842 T), a plant growth-promoting bacterium. J Bacteriol. 2011; 193:5026-7, for
P. polymyxa CF05 in Lei M, Lu P, Jin L, Wang Y, Qin J, Xu X, et al. Complete Genome Sequence of Paenibacillus polymyxa CF05, a Strain of Plant Growth-Promoting Rhizobacterium with Elicitation of Induced Systemic Resistance. Genome Announc. 2015; 3:2, for
P. polymyxa CR1 in Eastman A W, Weselowski B, Nathoo N, Yuan Z. Complete genome sequence of Paenibacillus polymyxa CR1, a plant growth-promoting bacterium isolated from the corn rhizosphere exhibiting potential for biocontrol, biomass degradation, and biofuel production. Genome Announcements. 2014; 2:1, for
P. polymyxa DSM 365 in Xie N Z, Li J X, Song L F, Hou J F, Guo L, Du Q S, et al. Genome sequence of type strain Paenibacillus polymyxa DSM 365, a highly efficient producer of optically active (R, R)-2,3-butanediol. J Biotechnol. 2015; 195:72-3, for
P. polymyxa E681 in Kim J F, Jeong H, Park S Y, Kim S B, Park Y K, Choi S K, et al. Genome sequence of the polymyxin-producing plant-probiotic rhizobacterium Paenibacillus polymyxa E681. J Bacteriol. 2010; 192:6103-4, for
P. polymyxa M-1 in Niu B, Rueckert C, Blom J, Wang Q, Borriss R. The genome of the plant growth-promoting rhizobacterium Paenibacillus polymyxa M-1 contains nine sites dedicated to nonribosomal synthesis of lipopeptides and polyketides. J Bacteriol. 2011; 193:5862-3, for
P. polymyxa NRRL B-30509 and P. terrae NRRL B-30644 in van Belkum M J, Lohans C T, Vederas J C. Draft Genome Sequences of Paenibacillus polymyxa NRRL B-30509 and Paenibacillus terrae NRRL B-30644, Strains from a Poultry Environment That Produce Tridecaptin A and Paenicidins. Genome Announc. 2015; 3:2, for.
P. polymyxa Sb3-1 in Rybakova D, Wetzlinger U, Muller H, Berg G. Complete Genome Sequence of Paenibacillus polymyxa Strain Sb3-1, a Soilborne Bacterium with Antagonistic Activity toward Plant Pathogens. Genome Announc. 2015; 3:2, for
P. polymyxa SC2 in Mingchao M, Wang C, Ding Y, Li L, Shen D, Jiang X, et al. Complete genome sequence of Paenibacillus polymyxa SC2, a strain of plant growth-promoting rhizobacterium with broad-spectrum antimicrobial activity. J Bacteriol. 2011; 193:311-2, for
P. polymyxa SQR-21 in Li S, Yang D, Qiu M, Shao J, Guo R, Shen B, et al. Complete genome sequence of Paenibacillus polymyxa SQR-21, a plant growth-promoting rhizobacterium with antifungal activity and rhizosphere colonization ability. Genome Announcements. 2014; 2:2, for
P. terrae HPL-003, in Shin S H, Kim S, Kim J Y, Song H Y, Cho S J, Kim D R, et al. Genome sequence of Paenibacillus terrae HPL-003, a xylanase-producing bacterium isolated from soil found in forest residue. J Bacteriol. 2012; 194:1266.

Fusaricidins A, B, C and D are reported to inhibit plant pathogenic fungi such as Fusarium oxysporum, Aspergillus niger, Aspergillus oryzae, and Penicillum thomii (J. Antibiotics 49(2), 129-135, 1996; J. Antibiotics 50(3), 220-228, 1997). Fusaricidins such as Li-F05, LI-F07 and LI-F08 have been found to have certain antifungal activity against various plant pathogenic fungi such as Fusarium moniliforme, F. oxysporum, F. roseum, Giberella fujkuroi, Helminthosporium sesamum and Penicillium expansum (J. Antibiotics 40(11), 1506-1514, 1987). In addition, fusaricidins have antifungal activity against Leptosphaeria maculans which causes black root rot of canola (Can. J. Microbiol. 48, 159-169, 2002).

Fusaricidins are mainly present in the spores of fusaricidin producing bacteria and seem to work mainly as contact fungicides. Accordingly, even spreading of fusaricidins and fusaridicin comprising bacterial spores on the plant surface is important to enhance their protective effect against plant infections. WO2017/151742 describe a method to harvest fusaricidin comprising bacterial spores in which an amphiphilic sulfonate and/or an amphiphilic sulfate is mixed with the cell culture as a centrifugation aid to form spore comprising aggregates, unwanted exopolysaccharides are separated from the centrifuges spores, and a polyoxyethylene glycol alkyl ether is added to the spores as an releasing aid to disrupt the aggregates.

Individual spores of fusaricidin producing bacteria and aggregates comprising such spores are particle-like substances. As a consequence, biologicals comprising spores, as individual spores and/or as aggregates comprising such spores, are often formulated in solid form, e.g as wettable powders or water-dispersible granules or dispersed in liquid formulation types, e.g. as aqueous or non-aqueous suspension concentrates or as oil dispersions.

Several formulation types for spores of lipopeptide producing bacteria and/or spores of fusaricidin producing bacteria have been developed:

WO2015/184170 discloses lauryl ether sulfates and other sulfate and sulfonate surfactants as synergistically active additives for lipopeptide-producing bacterial strains.

WO2017/210508 discloses non-aqueous—non-oil liquid Bacillus amyloliquefaciens compositions for use in the area of nematode control.

WO2009/126473 describe formulations for different Bacillus species comprising propylene glycol and further additives like methyl-methycrylate graft copolymers and sodium salts of lignosulfonic acids.

WO2013/010322 discloses Paenibacillus polymyxa wettable powder formulations with good physical properties like suspension rate, quick wetting and low wet sieve residues.

Different formulations for biologicals can greatly affect the storage time of biologicals, and therefore have to be optimized against unwanted effects like decomposition, agglomeration and/or the development of undesirable odor. It was one objective of the present invention to provide formulations of spores of fusaricidin producing bacteria that are stable and do not further agglomerate during storage or application.

However, different formulations can also influence the biological effectiveness of biologicals when applied to plants. Thus, it was one objective of the present invention to provide formulations for spores of fusaricidin producing bacteria, which show an enhanced effectiveness to prevent plant fungal infections and development of disease and show good application properties, e.g. for preparation of a water based spraying liquid and its application to plants. To a person skilled in the art of formulation of crop protection it is well known, that combinations of ionic with nonionic surface active compounds are necessary to provide physically stable formulations with acceptable application properties. The inventors found that many ionic dispersants and surfactants used as formulation additives in the field of biological and non-biological crop protection formulations, negatively interfere with the biological efficacy of spores of fusaricidin producing bacteria. Surprisingly, specific groups of nonionic surface-active additives, later called category 1 additives, could be identified which even highly promote the efficacy of water based spraying liquids with spores of fusaricidin producing bacteria to prevent or reduce fungal infections of plants. In addition, some ionic surface-active additives, later called category 2 additives, were identified, which do not inhibit the efficacy of spores of fusaricidin producing bacteria.

Moreover, the inventors identified, later called category 3 additives, which are detrimental to the efficacy of spores of fusaricidin producing bacteria when used alone but can positively contribute to the efficacy when used in combination with category 1 additives and/or category 2 additives. Such effective combinations of category 1 additives, category 2 additives and category 3 additives are disclosed herein.

Formulations and spraying liquids according to the invention comprise biomass comprising spores of at least one type of fusaricidin producing bacteria.

“Spores” as contemplated by the present invention refers to at least one dormant (at application) but viable reproductive unit of a fusaricidin producing bacterial species. It is further recognized that the spores disclosed herein are produced via culturing of fusaricidin producing bacteria and are usually harvested from the fermentation broth before used to prepare the formulations and spraying liquids of the invention. They can therefore comprise a combination of vegetative cells and forespores (cells in an intermediate stage of spore formation); a combination of forespores and spores; or a combination of forespores, vegetative cells and/or spores, as well as other solid components of the fermentation broth. Preferably the at least one spore, once applied to a plant and optionally after germination of the spore, will provide the plant with a known and proven agricultural benefit, such as but not excluding others an enhanced protection against fungal infection.

In one embodiment formulations and spraying liquids according to the invention comprise inorganic phosphate. The amount of inorganic phosphate in the spraying liquid is preferably selected to be between about 0.01 mM and about 1 M, preferably between about 0.1 mM and about 100 mM, when the spraying liquid is applied to the plant and/or the locus where the plant is growing. The inorganic phosphate usually comprises phosphoric acid, polyphosphoric acid, phosphorous acid and/or a salt of H₂PO₄⁻, H₂PO₃⁻, HPO₄²⁻ or PO₄³⁻. The inorganic phosphate may be provided by a tank mix with a fertilizer comprising inorganic phosphate, like Triple Superphosphate, a NPK fertilizer or rock phosphate or in a mixture with a solid formulation comprising the spores of at least one type of fusaricidin producing bacteria. In some embodiments, some or all of the inorganic phosphate is a component of the solid or liquid formulation used to prepare the spraying liquid. For example, solid formulations may comprise 2% to 30% w/w of inorganic phosphate, preferably liquid formulations comprise up to 10% w/w and solid formulations up to 30% w/w of inorganic phosphate.

In one embodiment, the spores of at least one type of fusaricidin producing bacteria are selected to be spores of a fusaricidin producing bacteria which has a high capacity to solubilize inorganic phosphate. One example for fusaricidin producing bacteria having a high capacity to solubilize inorganic phosphate is Paenibacillus polymyxa VMC10/96.

Fusaricidin producing bacterial species are all bacterial species which comprise a functional variant of a fusaricidin gene cluster. The general structure of a fusaricidin gene cluster has been published in Jingru Li and Susan E. Jensen Nonribosomal Biosynthesis of Fusaricidins by Paenibacillus polymyxa PKB1 Involves Direct Activation of a D-Amino Acid Chemistry & Biology 2008, 15, 118-127. Further variants of fusarididin gene cluster are described in the annotated genomes of Paenibacillus species Paenibacillus polymyxa strain E681, P. polymyxa ATCC 842 T, P. polymyxa CF05, P. polymyxa CR1, P. polymyxa DSM 365, P. polymyxa M-1, P. polymyxa NRRL B-30509, P. polymyxa Sb3-1, P. polymyxa SC2, P. polymyxa SQR-21, P. terrae NRRL B-30644, P. terrae HPL-003.

Fusaricidin producing bacterial species belong mainly to the genus Paenibacillus, including the species P. agarexedens, P. agaridevorans, P. alginolyticus, P. alkaliterrae, P. alvei, P. amylolyticus, P. anaericanus, P. antarcticus, P. assamensis, P. azoreducens, P. azotofixans, P. bardnonensis, P. borealis, P. brasiliensis, P. brassicae, P. campinasensis, P. chinjuensis, P. chitinolyticus, P. chondroitinus, P. cineris, P. cookie, P. curdlanolyticus, P. daejeonensis, P. dendritiformis, P. durum, P. ehimensis, P. elgii, P. favisporus, P. glucanolyticus, P. glycanilyticus, P. gordonae, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. nov. spec. epiphyticus, P. odorifer, P. pabuli, P. peoriae, P. phoenicis, P. phyllosphaerae, P. polymyxa, P. polymyxa ssp. polymyxa, P. polymyxa ssp. plantarum, P. popilliae, P. pulvifaciens, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii or P. xylanilyticus.

Preferred Paenibacillus species are Paenibacillus polymyxa, Paenibacillus polymyxa ssp. polymyxa, Paenibacillus polymyxa ssp. plantarum, Paenibacillus nov. spec. epiphyticus, Paenibacillus terrae, Paenibacillus macerans, and Paenibacillus alvei.

In one embodiment the Paenibacillus species are Paenibacillus polymyxa and Paenibacillus terrae. In one preferred embodiment, the spores belong to a specific strain of a Paenibacillus sp and its mutants or strain variants having at least 98.0%, 98.5%, 99.0%, 99.5%, 99.6%, 99.7%, 99.8%, 99.9% sequence identity, calculated based on the Average Nucleotide Identity method (ANI) for hierarchical classification (Konstantinidis, K. T. et al. (2005) PNAS USA 102(7):2567-72), and which preserve the capacity to produce fusaricidins. Preferred strains are Paenibacillus sp. strain Lu16774 deposited with DSMZ under Accession No. DSM 26969, Paenibacillus sp. strain Lu17007 deposited with DSMZ under Accession No. DSM 26970, Paenibacillus sp. strain Lu17015 deposited with DSMZ under Accession No. DSM 26971, Paenibacillus sp. strain NRRL B-50972, Paenibacillus sp. strain NRRL B-67129, Paenibacillus sp. strain NRRL B67304, Paenibacillus sp. strain NRRL B-67306, Paenibacillus sp. strain NRRL B-67615, Paenibacillus sp. strain NRRL B-50374, Paenibacillus sp. strain NRRL B-67721, Paenibacillus sp. strain NRRL B-67723, and Paenibacillus sp. strain NRRL B-67724, P. polymyxa strain 9.4E, P. polymyxa strain 10.6D, P. polymyxa strain VMC10/96, P. polymyxa strain KGS-3, Paenibacillus polymyxa ATCC 842 T, Paenibacillus polymyxa CR1, Paenibacillus polymyxa DSM 365, Paenibacillus polymyxa E681, Paenibacillus polymyxa M-1, Paenibacillus polymyxa Sb3-1, Paenibacillus polymyxa SC2, Paenibacillus polymyxa SQR-21 and Paenibacillus terrae HPL-003 or a fusaricidin producing mutant of one of these strains. Several of these strains have been disclosed in WO2014/092345, WO2016/020371, WO2016/154297, WO2016/187703, WO2016019480, WO2017/082761, WO2018/195603, WO2019/221988, WO2019/155253, WO202061140 and WO2020/154813.

Preferably the fermentation broth is concentrated to produce a “broth concentrate” by conventional industrial methods, as described above, but remains in liquid form and preferably washed, for example, via a diafiltration process, to remove residual fermentation broth and metabolites. This broth concentrate is then dried by methods used in the art, preferably it is dried via spray drying, to produce the biomass comprising spores of fusaricidin producing bacteria used to prepare the spraying liquids and formulations of the invention. The biomass comprising spores of fusaricidin producing bacteria has usually a residual water content of below 15% w/w, more preferably of below 10% w/w, most preferably of below 5% w/w. Because it is produced by drying a both concentrate, the biomass may also comprise a certain degree of remaining other solids of the fermentation broth. Preferably the biomass comprising spores of fusaricidin producing bacteria has an amount of 5×10⁸ to 1×10⁹ CFU/gram.

However, it is also possible to use both concentrates to prepare the liquid or solid formulations of the invention. In this case, the amount of biomass used for the solid or liquid formulations is calculated based on the amount of dry biomass of the respective concentrate. In case concentrates of the fermentation broth are used, the concentrates are preferably supplemented with a stabilizing agent selected from the group consisting of an amine, a quaternary ammonium compound, a salt of phosphate or citrate, a short chain polyol having from 2 to 10 carbon atoms, a urea, and combinations thereof as described in WO2019/222253.

Said bacterial spores and biomass are used in solid formulations or dispersed liquid formulations, which comprise category 1 additives and/or category 2 additives and may or may not comprise category 3 additives. Spraying liquids are prepared by adding water or oil to these solid or liquid formulations till the desired amount of spraying liquid per hectare, having the desired amount of biomass comprising spores of fusaricidin producing bacteria per hectare is reached. Spraying liquids can also be prepared by combining water with the contents of a kit of at least two parts, comprising the spores of fusaricidin producing bacteria in a first concentrated form and at least one of the category 1 additive and/or category 2 additive and/or category 3 additive in one or more further concentrated forms and wherein the relative amounts of the spores of fusaricidin producing bacteria and the relative amounts the category 1 additive and/or the category 2 additive and/or the category 3 additive in the kit are adapted to provide the amounts suitable to prepare the spraying liquids in the desired amount per hectare and having the desired amount of biomass comprising spores of fusaricidin producing bacteria per hectare.

Suitable category 1 additives are nonionic surface-active additives of the following groups:
1a) polyethylene glycol—polypropylene glycol block copolymers of the A-B-A type in which A is a hydrophilic block and B is a hydrophobic block (“Poloxamers”) or of the B-A-B type in which A is a hydrophilic block and B is a hydrophobic block (“Meroxapoles”). Preferred polyethylene glycol—polypropylene glycol block copolymers comprise polyethylene glycol-polypropylene glycolpolyethylene glycol block polymers with a molar mass of approximately 950 to 6500 g/mol and a polyethylene oxide content of 10% to 50% w/w;
preferably: poloxamers of the types polyethylene—polypropylene glycol block polymer with 10% ethyleneoxide, 850 g/mol such as but not excluding others Pluronic® PE3100, polyethylene—polypropylene glycol block polymer with 40% ethyleneoxide, 3250 g/mol such as but not excluding others Pluronic® PE 6400, polyethylene—polypropylene glycol block polymer with 20% ethyleneoxide, 3250 g/mol such as but not excluding others Pluronic® PE 6200 or polyethylene—polypropylene glycol block polymer with 50% ethyleneoxide, 1750 g/mol such as but not excluding others Pluronic® PE 10500;
even more preferred: polyethylene—polypropylene glycol block polymer with 40% ethyleneoxide, 3250 g/mol such as but not excluding others Pluronic® PE 6400, polyethylene—polypropylene glycol block polymer with 20% ethyleneoxide, 3250 g/mol such as but not excluding others Pluronic® PE 6200 or polyethylene—polypropylene glycol block polymer with 50% ethyleneoxide, 1750 g/mol such as but not excluding others Pluronic® PE 10500.

Poloxamers are nonionic triblock copolymers composed of a central hydrophobic chain of polyoxypropylene (poly(propylene oxide)) flanked by two hydrophilic chains of polyoxyethylene (poly(ethylene oxide)).

Because the lengths of the polymer blocks can be customized, many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mo} and a 70% polyoxyethylene content). For the Pluronic® and Synperonic® tradenames, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content (e.g., L61 indicates a polyoxypropylene molecular mass of 1800 g/mol and a 10% polyoxyethylene content). For example, poloxamer 184 (P184) equals Pluronic PE 6400 and Synperonic PE—L64.
1b) polyvinylpyrrolidines, polyvinylacetates, and statistical and block copolymers of polyvinyl pyrrolidone and polyvinyl acetate;
preferably: statistical and block copolymers of polyvinyl pyrrolidone and polyvinyl acetate, all with a molar mass of approximately 9000 to 70000 g/mol;
even more preferred: polyvinylpyrrolidone M 40 000 95% such as but not excluding others Sokalan® HP50 or polyvinylpyrrolidone-polyvinylacetate statistical copolymer such as but not excluding others Luvitec® VA 64.
1c) polyoxyethylene sorbitol fatty acid esters (“polysorbates”) and sorbitol fatty acid esters having a fatty acid chain with a length of 10 to 20 carbon atoms, preferably 12 to 18 carbon atoms, most preferably dodecanoic acid and oleic acid and linoleic acid, from 10 to 150 oxyethylene units, preferably 20 to 80 oxyethylene units, most preferred 20 to 40 oxyethylene units;
preferably: polyoxyethylene (40) sorbitol hexaoleate such as but not excluding others Atlas™ G-1086, sorbitol ethoxylate esters such as but not excluding others Atlas™ G-1096, poly oxyethylene (20) sorbitan monolaurate such as but not excluding others Tween® 20, poly(oxyethylene) (20) sorbitan monooleate such as but not excluding others Tween® 80, sorbitan monooleate (Span™ 80) or sorbitol ethoxylate ester of polymerized fatty acid such as but not excluding others Atlox™ 4916;
even more preferred: poly oxyethylene (20) sorbitan monolaurate such as but not excluding others Tween® 20, poly(oxyethylene) (20) sorbitan monooleate such as but not excluding others Tween® 80 and sorbitol ethoxylate ester of polymerized fatty acid such as but not excluding others Atlox™ 4916.
1d) fatty acid glycerol esters including fatty acid mono- and diesters of glycerol and polyglycerol, glycerolmonooleate, glyceroldioleate;
preferably: mixtures of polyglycerolesters and fatty acid esters such as but not excluding others Break-Thru® SP133 or glycerolmono/diooleat such as but not excluding others Break-Thru® EM O 5;
even more preferred: mixtures of polyglycerolesters and fatty acid esters such as but not excluding others Break-Thru® SP133.
1e) alcohol alcoxylates having from 5 to 80 oxyethylene units, wherein the alcohols are preferably C10-18 alcohols, more preferably C10-C13 alcohols, most preferred C10 alcohols, wherein the oxyethylene units are preferably 5 to 40 oxyethylene units more preferred from 7 to 14 oxyethylene units;
preferably: alcohol alcoxylates having from 5 to 40 oxyethylene units and wherein the alcohols are C10-C13 alcohols;
even more preferred: alcohol alcoxylates having from 7 to 14 oxyethylene units and wherein the alcohols are C10 alcohols;
most preferred: alkyl polyethylene glycol ether from C10-Guerbet alcohol, with an hydrophilic-lipophilic balance value (HLB) of 13 such as but not excluding others Lutensol® XL70, alkyl polyethylene glycol ether from C10-Guerbet alcohol, with an hydrophilic-lipophilic balance value (HLB) of 16 such as but not excluding others Lutensol® XL140, alkyl polyethylene glycol ether from C16 C18 fatty alcohol, with an hydrophilic-lipophilic balance value (HLB) of 18.5 such as but not excluding others Lutensol® AT 80 or polyoxyethylene (23) lauryl ether such as but not excluding others Brij®-23.
1f) alkyl glucosides having an alkyl chain length C5-C15 and 1 to 10 glucoside units,
preferably: alkyl polyglucosides having an alkyl chain length C8-C10 and mono- and diglucosides with an overall polymerization degree of 1.5,
even more preferred: Agnique® PG 8107-G

Suitable category 2 additives are ionic surface-active additives of the following groups:
2a) polyethylene glycol-grafted polyacrylates, and salts thereof, preferably sodium salts thereof;
preferably: methyl-methacrylate graft copolymer such as but not excluding others, Atlox™ 4913, and Tersperse® 2500.
2b) ethoxylated polyethyleneimines;
preferably: ethoxylated cationic polyethyleneimines such as but not excluding others Sokalan® HP20.
2c) sodium lauryl ether sulfates, preferably a sodium lauryl ether sulfate with 1 to 4 oxyethylene units and sodium dodecylpoly(oxyethylene)sulfates with 1 to 50 oxyethylene units, preferably with 1 to 7 oxyethylene units, most preferred with 1 to 4 oxyethylene units,
preferably: sodium lauryl ether sulfate such as but not excluding others Agnique® SLES 370, or Genapol® LRO).

Suitable category 3 additives are sulfonates, sulfonic acid co-condensates and sulfates of the following groups:
3a) lignosulfonates;
preferably: a lignosulfonic acid, ammonium salt, calcium salt or sodium salt such as but not excluding others Polyfon® H, Polyfon® F, Polyfon® O, Borresperse Ca, Borresperse Na, or Vanisperse® CB, even more preferred: Borresperse Na or Borresperse Ca,
3b) alkylbenzene sulfonates and salts thereof;
3c) alkylnaphthalene sulfonates and salts thereof;
3e) napthalene sulfonic acid co-condensates such as but not excluding others Wettol® D3;
3f) alkyl sulfonates and salts thereof;
3g) fatty alcohol sulfates and salts thereof, preferably sodium lauryl sulfate such as but not excluding others Tensiofix® BCZ, Loxanol® K12P or Teaxapon® K12.

Preferred groups are 3a) and 3g).

In some embodiments the spraying liquid, or the solid or liquid formulations comprise a mixture of several category 1 additives of groups 1a) to 1f).

Preferred additives for preparing such mixtures are one or more category 1 additives selected from the group consisting of:
a mixture of polyglycerolesters and fatty acid esters such as but not excluding others Break-Thru® SP133, a polyethylene—polypropylene glycol block polymer with 20% ethyleneoxide, 3250 g/mol such as but not excluding others Pluronic® PE 6200, a polyethylene—polypropylene glycol block polymer with 40% ethyleneoxide, 3250 g/mol such as but not excluding others Pluronic® PE 6400, a polyethylene—polypropylene glycol block polymer with 50% ethyleneoxide, 1750 g/mol such as but not excluding others Pluronic® PE 10500, a poly(oxyethylene) (20) sorbitan monooleate (Tween® 80), a sorbitan monooleate such as but not excluding others Span™ 80, and an alkyl polyglucoside such as but not excluding others Agnique® PG 8107-G, or

In some embodiments the spraying liquid, or the solid or liquid formulations comprise a mixture of one or more category 1 additives of groups 1a) to 1f) and one or more category 2 additives. Preferred additives for preparing such mixtures are one or more category 1 additives selected from the group consisting of:
a mixture of polyglycerolesters and fatty acid esters such as but not excluding others Break-Thru® SP133,
a polyethylene—polypropylene glycol block polymer with 20% ethyleneoxide, 3250 g/mol such as but not excluding others Pluronic® PE 6200,
a polyethylene—polypropylene glycol block polymer with 40% ethyleneoxide, 3250 g/mol such as but not excluding others Pluronic® PE 6400,
or a polyethylene—polypropylene glycol block polymer with 50% ethyleneoxide, 1750 g/mol such as but not excluding others Pluronic® PE 10500, and
an alkyl polyglucoside such as but not excluding others Agnique® PG 8107-G.

Preferred category 2 additives to be combined with one or more category 1 additives are category 2 additives selected from the group consisting of:
a methyl-methacrylate graft copolymer such as but not excluding others Atlox™ 4913 and a sodium laurylethersulfate such as but not excluding others Agnique® SLES 370 or Genapol® LRO.

Preferred combinations of category 1 additives which may or may not be combined with category 2 additives are
a. at least one polyethylene—polypropylene glycol block polymer with 50% ethyleneoxide, 1750 g/mol such as but not excluding others Pluronic® PE 10500 and at least one polyethylene—polypropylene glycol block polymer with 20% ethyleneoxide, 3250 g/mol such as but not excluding others Pluronic® PE 6200,
b. at least one polyethylene—polypropylene glycol block polymer with 50% ethyleneoxide, 1750 g/mol such as but not excluding others Pluronic® PE 10500) and at least one mixture of polyglycerolesters and fatty acid esters such as but not excluding others Break-Thru® SP133,
c. at least one polyethylene—polypropylene glycol block polymer with 50% ethyleneoxide, 1750 g/mol such as but not excluding others Pluronic® PE 10500 and at least one polyethylene—polypropylene glycol block polymer with 40% ethyleneoxide, 3250 g/mol such as but not excluding others Pluronic® PE 6400,
d. at least one poly(oxyethylene) (20) sorbitan monooleate such as but not excluding others Tween® 80 and at least one sorbitan monooleate such as but not excluding others Span™ 80, or
e. at least one polyethylene—polypropylene glycol block polymer with 10% ethyleneoxide, 850 g/mol such as but not excluding others Pluronic® PE3100 and at least one a polyvinylpyrrolidone with an average molar mass of maximally 50 000 g/mol such as but not excluding others Sokalan® HP 50.

Preferred combinations of one or more category 1 additives and one or more category 2 additives are:
a. at least one polyethylene—polypropylene glycol block polymer with 50% ethyleneoxide, 1750 g/mol such as but not excluding others Pluronic® PE 10500 and at least one polyethylene—polypropylene glycol block polymer with 20% ethyleneoxide, 3250 g/mol such as but not excluding others Pluronic® PE 6200 as category 1 additives and at least one sodium laurylethersulfate such as but not excluding others Agnique® SLES 370 or Genapol® LRO as category 2 additive
b. at least one polyethylene—polypropylene glycol block polymer with 20% ethyleneoxide, 3250 g/mol such as but not excluding others Pluronic® PE 6200 as category 1 additive and at least one methyl-methacrylate graft copolymer such as but not excluding others Atlox™ 4913 as category 2 additive,
c. at least one polyethylene—polypropylene glycol block polymer with 50% ethyleneoxide, 1750 g/mol such as but not excluding others Pluronic® PE 10500 as category 1 additive and at least one methyl-methacrylate graft copolymer such as but not excluding others Atlox™ 4913 as category 2 additive,
d. at least one polyethylene—polypropylene glycol block polymer with 50% ethyleneoxide, 1750 g/mol such as but not excluding others Pluronic® PE 10500 and at least one mixture of polyglycerolesters and fatty acid esters such as but not excluding others Break-Thru® SP133 as category 1 additives and at least one methyl-methacrylate graft copolymer such as but not excluding others Atlox™ 4913 as category 2 additive,
e. at least one polyethylene—polypropylene glycol block polymer with 50% ethyleneoxide, 1750 g/mol such as but not excluding others Pluronic® PE 10500 and at least one polyethylene—polypropylene glycol block polymer with 40% ethyleneoxide, 3250 g/mol such as but not excluding others Pluronic® PE 6400 as category 1 additives and at least one methyl-methacrylate graft copolymer such as but not excluding others Atlox™ 4913 as category 2 additive,
f. at least one polyethylene—polypropylene glycol block polymer with 40% ethyleneoxide, 3250 g/mol such as but not excluding others Pluronic® PE 6400 as category 1 additive and at least one methyl-methacrylate graft copolymer such as but not excluding others Atlox™ 4913 as category 2 additive, or
g. at least one alkyl polyglucoside such as but not excluding others Agnique® PG 8107-G as category 1 additive and at least one methyl-methacrylate graft copolymer such as but not excluding others Atlox™ 4913 as category 2 additive.

In some embodiments the spraying liquids, or the solid or liquid formulations comprise a mixture of one or several category 1 additives of groups 1a) to 1f) and none, one or more category 2 additives and one or more category 3 additives selected from the group of
3a) lignosulfonates;
3b) alkylbenzene sulfonates and salts thereof;
3c) alkylnaphthalene sulfonates and salts thereof;
3e) napthalene sulfonic acid co-condensates;
3f) alkyl sulfonates and salts thereof;
3g) fatty alcohol sulfates and salts thereof, preferably sodium lauryl sulfate.

The total amount of biomass comprising spores of fusaricidin producing bacteria in spraying liquids is preferably between 0.005% w/w and 2% w/w, 0.0167% w/w and 2% w/w, 0.0375% w/w and 2% w/w, 0.1500% w/w and 2% w/w, or 0.4000% w/w and 2% w/w or between 0.005% w/w and 0.4000% w/w, 0.0167% w/w and 0.4000% w/w, 0.0375% w/w and 0.4000% w/w, 0.1500% w/w and 0.4000% w/w, or between 0.0167% w/w and 0.4000% w/w.

The total amount of category 1 and category 2 additives in spraying liquids is preferably between 0.0020% w/w and 2% w/w, 0.0117% w/w and 2% w/w, 0.0250% w/w and 2% w/w, 0.1500% w/w and 2% w/w or 0.4% w/w and 2% w/w, or between 0.0020% w/w and 0.4% w/w, 0.0117% w/w and 0.4% w/w, 0.0250% w/w and 0.4% w/w, 0.1500% w/w and 0.4% w/w, or between 0.0117% w/w and 0.1500% w/w.

If present, the total amount of category 3 additives in spraying liquids is preferably between 0.0001% w/w and 0.08% w/w, 0.0003% w/w and 0.08% w/w, 0.0013% w/w and 0.08% w/w, or 0.001% w/w and 0.08% w/w, or between 0.0001% w/w and 0.001% w/w, 0.0003% w/w and 0.001% w/w, 0.0013% w/w and 0.001% w/w, or between 0001% w/w and 0.0013% w/w, 0.0003% w/w and 0.0013% w/w. In some embodiments no category 3 additive is present.

If present, the total amount of category 3 additives in spraying liquids is usually higher, if the spraying liquids comprise a high amount of biomass comprising spores of fusaricidin producing bacteria. Preferably, the ratio of the amount of category 3 additives to the amount of biomass comprising spores of fusaricidin producing bacteria is preferably less than, in a rising degree of preference, 1:15, 1:16, 1:17. 1:18, 1:19, 1:20.

The total amount of biomass comprising spores of fusaricidin producing bacteria in solid or liquid formulations, preferably wettable powder (WP) or suspension concentrate (SC) formulations, is preferably between 5% w/w and 50% w/w, 10% w/w and 50% w/w, 15% w/w and 50% w/w, 30% w/w and 50% w/w, or 40% w/w and 50% w/w, or between 5% w/w and 40% w/w, 10% w/w and 40% w/w, 15% w/w and 40% w/w, 30% w/w and 40% w/w, or between 10% w/w and 40% w/w, 15% w/w and 40% w/w, 30% w/w and 40% w/w

The total amount of category 1 and category 2 additives in solid or liquid formulations, preferably wettable powder (WP) or suspension concentrate (SC) formulations, is preferably between 2% w/w and 50% w/w, 7% w/w and 50% w/w, 10% w/w and 50% w/w, 30% w/w and 50% w/w, 40% w/w and 50% w/w, or between 2% w/w and 40% w/w, 7% w/w and 40% w/w, 10% w/w and 40% w/w, 30% w/w and 40% w/w, or between 7% w/w and 40% w/w, 10% w/w and 40% w/w, 30% w/w and 40% w/w.

If present, the total amount of category 3 additives in solid or liquid formulations, preferably wettable powder (WP) or suspension concentrate (SC) formulations, is preferably between 0.05% w/w and 2% w/w, 0.1% w/w and 2% w/w, 0.5% w/w and 2% w/w, or 1% w/w and 2% w/w or between 0.05% w/w and 0.5% w/w, 0.1% w/w and 0.5% w/w, 0.5% w/w and 0.5% w/w, or between 0.05% w/w and 0.5% w/w, 0.1% w/w and 0.5% w/w.

If present, the total amount of category 3 additives in spraying liquids is usually higher, in the solid or liquid formulations comprising a high amount of biomass comprising spores of fusaricidin producing bacteria. Preferably, the ratio of the amount of category 3 additives to the amount of biomass comprising spores of fusaricidin producing bacteria is preferably less than, in a rising degree of preference, 1:15, 1:16, 1:17, 1:18, 1:19, 1:20.

Usually the total amount of solid or liquid formulations per hectare is between 0.5 kg/ha and 4 kg/ha, 1 kg/ha and 4 kg/ha, 2 kg/ha and 4 kg/ha or between 0.5 kg/ha and 2 kg/ha, 1 kg/ha and 2 kg/ha, preferably the total amount is between 1 kg/ha and 2 kg/ha.

When preparing spraying liquids, the desired amount of biomass comprising spores of fusaricidin producing bacteria per hectare is between 50 g/ha and 2000 g/ha, 100 g/ha and 2000 g/ha, 150 g/ha and 2000 g/ha, 600 g/ha and 2000 g/ha or 800 g/ha and 2000 g/ha or between 50 g/ha and 1000 g/ha, 100 g/ha and 1000 g/ha, 150 g/ha and 1000 g/ha, 600 g/ha and 1000 g/ha or 800 g/ha and 1000 g/ha, or between 50 g/ha and 800 g/ha, 100 g/ha and 800 g/ha, 150 g/ha and 800 g/ha, 600 g/ha and 800 g/ha or between 150 g/ha and 1000 g/ha, 150 g/ha and 1000 g/ha, 600 g/ha and 1000 g/ha.

The desired amount of category 1 and category 2 additives in spraying liquids per hectare is between 20 g/ha and 2000 g/ha, 70 g/ha and 2000 g/ha, 100 g/ha and 2000 g/ha, 600 g/ha and 2000 g/ha, 800 g/ha and 2000 g/ha, or between 20 g/ha and 800 g/ha, 70 g/ha and 800 g/ha, 100 g/ha and 800 g/ha, or between 20 g/ha and 600 g/ha, 70 g/ha and 600 g/ha, 100 g/ha and 600 g/ha.

If present, the total amount of category 3 additives in in spraying liquids per hectare is between 0.5 g/ha and 80 g/ha, 1 g/ha and 80 g/ha, 5 g/ha and 80 g/ha, 20 g/ha and 80 g/ha or between 0.5 g/ha and 20 g/ha, 1 g/ha and 20 g/ha, 5 g/ha and 20 g/ha, or between 0.5 g/ha and 5 g/ha, 1 g/ha and 5 g/ha.

Usually the total amount of spraying liquids per hectare is between 1000 L/ha and 100 L/ha, 600 L/ha and 100 L/ha, 400 L/ha and 100 L/ha, 200 L/ha and 100 L/ha or between 1000 L/ha and 600 L/ha, 1000 L/ha and 400 L/ha or 1000 L/ha and 200 L/ha, or between 600 L/ha and 200 L/ha, 600 L/ha and 400 L/ha.

Wherein a higher amount of spraying liquid per hectare usually correlates with an even and complete coverage of the plants surface. The total amount of spraying liquids per hectare is usually prepared by adding the solid or liquid formulations in the amounts chosen to be applied per hectare to water or oil or a mixture of water and oil, and preferably to water.

The spraying liquids can also be prepared by using a kit of at least two parts, wherein the kit of parts comprises one or several parts wherein the contents of these parts, provide, when combined, the biomass comprising spores of fusaricidin producing bacteria, the category 1, category 2 and, if present, the category 3 additive in amounts as required to produce a spraying liquid comprising a total amount of biomass comprising spores of fusaricidin producing bacteria between 0.005% w/w and 2% w/w, 0.0167% w/w and 2% w/w, 0.0375% w/w and 2% w/w, 0.15% w/w and 2% w/w, 0.4% w/w and 2% w/w, a total amount of category 1 and category 2 additives preferably between 0.002% w/w and 2% w/w, 0.0117% w/w and 2% w/w, 0.025% w/w and 2% w/w, 0.15% w/w and 2% w/w, 0.4% w/w and 2% w/w and, if required, a total amount of category 3 additives preferably between 0.0001% w/w and 0.08% w/w, 0.0003% w/w and 0.08% w/w, 0.0013% w/w and 0.08% w/w, 0.001% w/w and 0.08% w/w.

Suitable customary types of solid or liquid formulations are suspensions, dusts, powders, pastes, granules, pressings, capsules, and mixtures thereof. Examples for formulation types are suspensions (e. g. SC, OD, FS), capsules (e. g. CS, ZC), pastes, pastilles, wettable powders, water dispersible powders, oil dispersible powders or dustable powders (e. g. WP, WS, OP, DP), pressings (e. g. BR, TB, DT, WT), water dispersible or direct applied granules (e. g. WG, GR), as well as gel formulations for the treatment of plant propagation materials such as seeds (e. g. GF).

Herein, it has to be taken into account that each formulation type or choice or selected auxiliary should not influence the viability of the microorganism during storage of the formulation and when finally applied to the soil, plant or plant propagation material. These and further formulation types are defined in the “Catalogue of pesticide formulation types and international coding system”, Technical Monograph No. 2, 6^{th} Ed. May 2008, CropLife International.

The formulations are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005. Solid formulations, in particular particulate formulations, like granules or powders are preferably provided in vacuum sealed multifoil or mylar pouches, or pouches made of water-soluble polymers, e.g. polyvinyl alcohol pouches.

Examples of formulation types and methods for their preparation are:

### i) Suspensions (SC, OD, FS)

In an agitated ball mill or colloid mill, 20-60 wt % of dried and ground biomass of fusaricidin producing bacteria of the invention are comminuted with addition of 5% w/w to 50% w/w of at least one category 1 additive or at least one category 2 additive or a mixture of at least one category 1 additive or at least one category 2 additive and less than 2% w/w of a category 3 additive, 5% w/w to 50% w/w of at least one water-soluble organic liquid, preferably also 0.1 to 2% w/w thickener (e. g. xanthan gum), and water ad 100% w/w to give a fine active substance suspension. Dilution with water gives a stable suspension of the active substance. For FS type composition up to 40% w/w binder (e. g. polyvinyl alcohol) is added. For water free suspensions, the amount of water at 100% w/w, can be replaced with additional water-soluble organic liquid. oil-dispersions (OD) can be produced by mixing spray-dried technical active ingredient (biomass) with oil, at least one category 1 additive or at least one category 2 additive or a mixture of at least one category 1 additive or at least one category 2 additive and less than 2% w/w of a category 3 and optionally further auxiliaries like emulsifiers, anti-foaming agents and thickening agents.

### ii) Water-Dispersible Granules (WG)

50-80 wt % of biomass of fusaricidin producing bacteria are ground finely with addition of 5% w/w to 50% w/w of at least one category 1 additive or at least one category 2 additive or a mixture of at least one category 1 additive or at least one category 2 additive and less than 2% w/w of a category 3 additive ad 100 wt % and prepared as water-dispersible or water-soluble granules by means of technical appliances (e. g. extrusion, spray tower, fluidized bed, preferably fluidized-bed-granulation). Dilution with water gives a stable dispersion or solution of the active substance. Another way of producing water-dispersible granules is by mixing a wet dough from biomass of fusaricidin producing bacteria with at least one category 1 additive or at least one category 2 additive or a mixture of at least one category 1 additive or at least one category 2 additive and less than 2% w/w of a category 3 additive, optionally fillers, and passaging the dough through a low-pressure dome extruder, followed by a drying step, e.g. by fluidized bed drying to form the granules.

### ii) Wettable Powders (WP)

50-80 wt % of dried and ground biomass of fusaricidin producing bacteria is mixed with addition of 5% w/w to 50% w/w of at least one category 1 additive or at least one category 2 additive or a mixture of at least one category 1 additive or at least one category 2 additive and less than 2% w/w of a category 3 additive and solid carrier (e. g. silica, diatomaceous earth, kaolin, soluble inorganic salts, sugars, maltodextrin) ad 100% w/w. Alternatively, the one category 1 additive or at least one category 2 additive or a mixture of at least one category 1 additive or at least one category 2 additive and less than 2% w/w of a category 3 additive, optionally fillers can be added before the drying step. Drying can be carried out by spray-drying to yield a fine powder. Dilution with water gives a stable dispersion or solution of the active substance.

iii) Dustable Powders (DP, DS)

1 to 10% w/w of biomass of fusaricidin producing bacteria is ground finely and mixed intimately with solid carrier (e. g. finely divided kaolin) ad 100% w/w.

### iv) Granules (GR, FG)

0.5 to 30% w/w of biomass of fusaricidin producing bacteria is ground finely and associated with solid carrier (e. g. silicate) ad 100% w/w. Granulation is achieved by extrusion, spray-drying or in a fluidized bed.

### v) Ultra-Low Volume Liquids (UL)

1 to 50% w/w of biomass of fusaricidin producing bacteria is dissolved in organic solvent (e. g. aromatic hydrocarbon) ad 100% w/w.

The compositions types i) to v) may optionally comprise further auxiliaries, such as 0.1 to 1% w/w bactericides, 5 to 15% w/w anti-freezing agents, 0.1 to 1.5% w/w anti-foaming agents, and 0.1 to 20% w/w colorants.

The compositions types iv to v are preferably used as a component of a kit of at least two parts in which the formulations iv to vii represent the first concentrated form and at least one of the category 1 additive and/or category 2 additive and/or category 3 additive are provided in at least one further concentrated form.

Formulations for seed treatment, i.e. suspoemulsions (SE), flowable concentrates (FS), powders for dry treatment (DS), water-dispersible powders for slurry treatment (WS), are usually employed for the purposes of treatment of plant propagation materials, particularly seeds.

Preferred examples of seed treatment formulation types or soil application for pre-mix compositions are of WS, FS or WG-type.

Further auxiliaries may be added to prepare the formulations, such auxiliaries are soluble fillers, carriers, insoluble fillers, thickening agents, rheology modifiers and anticaking agents, antifoaming agents, colorants, but also pH modifiers, pH buffers, biocides, adhesion agents, humectants, repellents, attractants, compatibilizers, preservatives, tackifiers and binders. Suitable soluble fillers, carriers, insoluble fillers, thickening agents, rheology modifiers and anticaking agents, anti-foaming agents, colorants, but also buffers, pH modifiers, biocides, adhesion agents, humectants, repellents, attractants, compatibilizers, preservatives, tackifiers and binders are well known in the art. Category 1 additives, category 2 additives and category 3 additives are excluded from the group of further auxiliaries.

Preferred soluble fillers or carriers, are inorganic salts like ammonium sulfate, magnesium sulfate, dibasic or monobasic potassium or sodium phosphate, or soluble organic compounds like lactose, sucrose, maltose, fructose, maltodextrin, soluble modified starch;

Preferred insoluble fillers are chalk, calcium carbonate, clays, kaolin, diatomaceous earth, precipitated or fumed silica.

Preferred thickening agents, rheology modifiers and anticaking agents are clays and organoclays, hydrophilic and hydrophobic silicas, soluble polymers such as polyvinylpyrrolidones and block copolymers of ethylene oxide and propylene oxide, xanthan gum or modified cellulose such as microcrystalline cellulose and hydroxypropyl cellulose;

Preferred anti-foaming agents, are silicone antifoaming agents, long chain alcohols, salts of fatty acids;

Preferred colorants (e.g. in red, blue, or green) are as pigments of low water solubility and water-soluble dyes including but not limited to inorganic colorants (e.g. iron oxide, titan oxide, iron hexacyanoferrate) and organic colorants (e.g. alizarin-, azo- and phthalocyanine colorants).

In order to avoid growth of the microorganisms of the invention or other organisms during storage, the solid or liquid formulations may comprise one or more biostatic agents as further additives. Suitable biostatic agents are small organic acids, lactic acid, acetic acid, sorbic acid, ascorbic acid, benzoic acid and salts thereof. Another method to prevent microorganism growth during storage is lowering the water content below 40% w/w, preferably below 10% w/w, most preferably below 5% w/w.

The spores of the fusaricidin producing bacteria are usually present in the formulations in the form of solid particles having a particle size d50 (50% of particles have a diameter below this value) between 1 to 25 μm, preferably 1 to 10 μm, more preferably 1 to 8 μm and a particle size d90 (90% of particles have a diameter below this value)between 1 to 90 μm, preferably 1 to 80 μm, more preferably 1 to 70 μm, and even more preferred 1 to 60 μm (determined according to light scattering method in liquid dispersion according to CIPAC method 187). Preferably the average particle size is in these areas even after a long time of storage, like storage for 16 weeks at room temperature or even after storage for 16 weeks at 40° C.

The density number of spores per ml can be determined by identifying the number of colony forming units (CFU) on agar medium e. g. potato dextrose agar after incubation for several days at temperatures of about 20 to about 35° C.

The amount of CFU/g of biomass used to prepare the formulations of the invention are usually between 1×10⁸ CFU/g bis 1×10¹⁰ CFU/g, preferably between 5×10⁸ to 5×10⁹ CFU/g. In one embodiment it is between 5×10⁸ to 1×10⁹ CFU/g The CFU/g of biomass will influence the amount of biomass which is used to prepare the formulations of the invention. Biomass having a comparatively high amount of CFU/g can be used to prepare formulations having a comparatively low amount of biomass. The amount of biomass used for preparing the formulations of the invention is usually selected to fit the amount of CFU per hectare, which should be applied for the respective purpose.

The amount of the other components of the respective formulations, like category 1 additives, category 2 additives and, if present, category 3 additives and the amount of further additives are selected to achieve further desired features, like storage stability, low viscosity, and good dilution properties, like re-dispersibility, wettability, dispersibility and suspensibility, and stability of the spraying liquid.

Liquid formulations according to the invention preferably have a viscosity of 80 to 350 mPas at a shear rate of 100 s⁻¹ (rotational rheometer acc. CIPAC method 192). Preferably the liquid formulations have a viscosity of 80 to 350 mPas at a shear rate of 100 s-′ even after storage for 16 weeks at room temperature or after storage for 16 weeks at 40° C.

In case in any of the formulations given herein the amount of the shares of each component exceeds 100%, the maximum amount of the continuous phase shall be reduced accordingly such that the amount of the shares of each component equals 100 wt %.

In some embodiments, a solid formulation, preferably a wettable powder (WP) formulation, has the following components.
Component 1: biomass comprising spores of fusaricidin producing bacteria
Component 2: an insoluble filler, preferably silica, a silicate or calcium carbonate or a mixture of both
Component 3: ammonium sulfate, sodium sulfate or magnesium sulfate (w/o water), or a mixture of them, or a mixture of hydrogenphosphate and dihydrogenphosphate sodium or potassium salt
Component 4: a category 1 additive, preferably from the group 1a, more preferred selected from Pluronic® PE 10500 or Pluronic® PE 6200 and most preferred being a combination of Pluronic® PE 10500 and Pluronic® PE 6200.
and optionally a
Component 5, being a category 2 additive, preferably from the group 2c, more preferred, being Agnique® SLES 370 or Genapol® LRO.

Wherein the different components 1 to 4 and optionally 5 are present in the amounts of:
Component 1: 20 to 40% w/w
Component 2: 10 to 40% w/w
Component 3: 0 to 25% w/w
Component 4: 15 to 40% w/w
Component 5: 0.5 to 1.9% w/w
   wherein the amounts of components 1 to 5 and, optionally, auxiliaries are selected to add up to 100% w/w.

Preferably the different components 1 to 4 and optionally 5 are present in the amounts of:
Component 1: 24 to 38% w/w
Component 2: 15 to 38% w/w
Component 3: 5 to 25% w/w
Component 4: 16 to 25% w/w
Component 5: 0.8 to 1.5% w/w.
   wherein the amounts of components 1 to 5 and, optionally, auxiliaries are selected to add up to 100% w/w.

More preferred the different components 1 to 4 and optionally 5 are present in the amounts of:
Component 1: 27 to 33% w/w
Component 2: 20 to 33% w/w
Component 3: 10 to 22% w/w
Component 4: 18 to 22% w/w
Component 5: 0.8 to 1.3% w/w.
   wherein the amounts of components 1 to 5 and, optionally, auxiliaries are selected to add up to 100% w/w.

Even more preferred amounts of components 1 to 4 and 5, if present, are disclosed in the examples described herein.

In some embodiments, a solid formulation, preferably a wettable powder (WP) formulation, comprises the following components.
Component 1: biomass comprising spores of fusaricidin producing bacteria
Component 2: an insoluble filler, preferably silica, a silicate or calcium carbonate or a mixture of both
Component 3: ammonium sulfate, sodium sulfate or magnesium sulfate (w/o water), or a mixture of them, or a mixture of hydrogenphosphate and dihydrogenphosphate sodium or potassium salt, preferably ammonium sulfate,
Component 4: a wetting agent other than a category 1, category 2 or category 3 additive, preferably a siloxane wetting agent, even more preferred being Break Thru S 301
Component 5: a category 1 additive, preferably from the group 1a or 1d, more preferred selected from Pluronic® PE 10500, Pluronic® PE 6200 or Break Thru SP 133 and most preferred being a combination of Pluronic® PE 10500 and Pluronic® PE 6200 or a combination of Pluronic® PE 10500 and Break Thru SP 133.
and preferably also a
Component 6, being a category 2 additive, preferably from the group 2a, more preferred, being Atlox™ 4913.

Optionally the solid formulation, preferably a wettable powder (WP) formulation, can comprise further auxiliaries. Further suitable auxiliaries are preferably soluble fillers, pH buffers and drying aids. These auxiliaries are applied in the amounts usually used in the art.

Wherein the different components 1 to 6 are present in the amounts of:
Component 1: 20 to 40% w/w
Component 2: 10 to 40% w/w
Component 3: 0 to 25% w/w
Component 4: 0.5 to 5% w/w
Component 5: 10 to 25% w/w
Component 6: 1 to 15% w/w.
   wherein the amounts of components 1 to 5 and, optionally, auxiliaries are selected to add up to 100% w/w.

Preferably the different components 1 to 6 are present in the amounts of:
Component 1: 24 to 38% w/w
Component 2: 15 to 38% w/w
Component 3: 5 to 25% w/w
Component 4: 1 to 5% w/w
Component 5: 10 to 25% w/w.
Component 6: 1 to 10% w/w.
   wherein the amounts of components 1 to 5 and, optionally, auxiliaries are selected to add up to 100% w/w.

More preferred the different components 1 to 6 are present in the amounts of:
Component 1: 27 to 33% w/w
Component 2: 27 to 33% w/w
Component 3: 18 to 22% w/w
Component 4: 2 to 3% w/w
Component 5: 10 to 25% w/w
Component 6: 5 to 8% w/w.
   wherein the amounts of components 1 to 5 and, optionally, auxiliaries are selected to add up to 100% w/w.

Even more preferred amounts of components 1 to 4 and 5, if present, are disclosed in the examples described herein.

In some embodiments, a liquid formulation, preferably a suspension concentrate (SC) formulation, comprises the following components.
Component 1: biomass comprising spores of fusaricidin producing bacteria
Component 2: a water-soluble organic liquid, preferably glycerol, propane-1,2-diol (propylen glycol), butane-1,4-diol, bu-tane-1,3-diol, pentane-1,5-diol, hexane-1,6-diol-2-methyl-2,4-pentanediol (hexylene glycol), heptan-1,7-diol, 2-ethylhexan-1,3-diol, polyethylene glycol (PEG) with a mean molecular weight of 150 g/mol to 450 g/mol, polypropylene glycol (PPG) with a mean molecular weight of 200 to 500 g/mol, propylene glycol methyl ether, dipropylene glycol methyl ether, tripropylene glycol methyl ether, dipropylene glycol dimethyl ether, propylene glycol n-propyl ether, diethylene glycol monobutylether, 2-hydroxypropane-1,2,3-tricarboxylate (triethyl citrate) N,N-dimethyl lactamide or a mixture of at least two or these, more preferred polyethylene glycol (PEG) with a mean molecular weight of 150 g/mol to 450 g/mol, propane-1,2-diol, 2-methyl-2,4-pentanediol or triethyl citrate or a mixture of these, more preferred triethyl citrate, propane-1,2-diol (propylene glycol), 2-methyl-2,4-pentanediol (hexylene glycol), PEG 400 and PEG 200 or a mixture of at least two of these, even more preferred being PEG 200,
Component 3: a category 1 additive, preferably from the group 1a or 1d, more preferred selected from Pluronic® PE 10500, Pluronic® PE 6200 or Break Thru SP 133 and most preferred being a combination of Pluronic® PE 10500 and Pluronic® PE 6200 or being a combination of Pluronic® PE 10500 and Break Thru SP 133.
Component 4, a category 2 additive, preferably from the group 2a, more preferred, being Atlox™ 4913
Component 5: water.

Optionally the liquid formulation, preferably a suspension concentrate (SC) formulation, can comprise further auxiliaries. Preferred further auxiliaries are tickening agents, antifoaming agents, preservatives, biostatic agents, and pH buffers. These auxiliaries are applied in the amounts usually used in the art.

Wherein the different components 1 to 4 and optionally 5 are present in the amounts of:
Component 1: 50 to 300 g/L
Component 2: 250 to 500 g/L
Component 3: 10 to 150 g/L
Component 4: 5 to 150 g/L
Component 5: 0 to 685 g/L
   wherein the amounts of components 1 to 5 and, optionally, auxiliaries are selected to add up to 1 Liter.

Preferably the different components 1 to 4 and optionally 5 are present in the amounts of:
Component 1: 100 to 250 g/L
Component 2: 300 to 450 g/L
Component 3: 20 to 130 g/L
Component 4: 10 to 100 g/L
Component 5: 0 to 570 g/L
   wherein the amounts of components 1 to 5 and, optionally, auxiliaries are selected to add up to 1 Liter.

More preferred the different components 1 to 4 and optionally 5 are present in the amounts of:
Component 1: 180 to 220 g/L
Component 2: 380 to 420 g/L
Component 3: 40 to 110 g/L
Component 4: 15 to 80 g/L
Component 5: 250 to 385 g/L
   wherein the amounts of components 1 to 5 and, optionally, auxiliaries, are selected to add up to 1 Liter.

Even more preferred amounts of components 1 to 4 and 5, if present, are disclosed in the examples described herein.

Alternatively, the liquid formulation, preferably a suspension concentrate (SC) formulation may comprise broth concentrate comprising biomass comprising spores of fusaricidin producing bacteria. Such liquid formulations comprise the following components:
Component 1: broth concentrate comprising biomass comprising spores of fusaricidin producing bacteria
Component 2: at least one category 1 additive, preferably Pluronic PE 10500, Pluronic PE 6200, Pluronic PE 6400 or Pluronic PE 3100
Component 3: a category 2 additive, preferably Atlox 4913, and
optionally Component 4: a stabilizer and/or further auxiliaries.

More preferred, the different components 1 to 3 are present in the amounts of:
Component 1: 4% w/w to 50% w/w
Component 2: 1% w/w to 20% w/w
Component 3: 2% w/w to 10% w/w
Component 4: 20% w/w to 93% w/w.
   Wherein the amounts of components 1 to 4 are selected to add up to 100% w/w.

The stabilizer of Component 4 is preferably a soluble salt like calcium nitrate, calcium chloride, sodium chloride, potassium chloride and/or a salt of small organic acids like sorbic acid, lactic acid, benzoic acid, and

In some embodiments, a liquid formulation, preferably a suspension concentrate (SC) formulation, comprises the following components.
Component 1: biomass comprising spores of fusaricidin producing bacteria
Component 2: a water-soluble organic liquid, glycerol, propane-1,2-diol (propylen glycol), butane-1,4-diol, butane-1,3-diol, pentane-1,5-diol, hexane-1,6-diol, 2-methyl-2,4-pentanediol (hexylene glycol), polyethylene glycol (PEG) with a mean molecular weight of 150 g/mol to 450 g/mol, polypropylene glycol (PPG) with a mean molecular weight of a mean molecular weight of 200 to 500 g/mol, propylene glycol methyl ether, dipropylene glycol methyl ether, tripropylene glycol methyl ether, dipropylene glycol dimethyl ether, propylene glycol n-propyl ether, diethylene glycol monobutylether, triethyl 2-hydroxypropane-1,2,3-tricarboxylate (triethyl citrate), tributyl 2-hydroxypropane-1,2,3-tricarboxylat (tributyl citrate), N,N-dimethyl lactamide or a mixture of these, more preferred polyethylene glycol (PEG) with a mean molecular weight of 150 g/mol to 450 g/mol, propane-1,2-diol, 2-methyl-2,4-pentanediol, triethyl citrate or tributyl citrate or a mixture of at least two of these, more preferred triethyl citrate propane-1,2-diol (propylen glycol), 2-methyl-2,4-pentanediol (hexylene glycol), PEG 400 and PEG 200 or a mixture of at least two of these, most preferred PEG 200,
Component 3: an anti-settling agent, preferably selected from clays and organoclays, hydrophilic and hydrophobic silicas, more preferred being Aerosil 200,
Component 4, a category 1 additive, preferably from the group 1a or 1f, more preferred selected from Pluronic® PE 6400 and Agnique® PG 8107 or being a combination of Pluronic® PE 6400 and Agnique® PG 8107.
Component 4, a category 2 additive, preferably from the group 2a, more preferred being Atlox™ 4913,

Optionally the liquid formulation, preferably a non-aqueous suspension concentrate (SC) formulation, can comprise auxiliaries. Preferred further auxiliaries are tickening agents, antifoaming agents, preservatives, biostatic agents, and pH buffers. These auxiliaries are applied in the amounts usually used in the art.

Wherein the different components 1 to 4 are present in the amounts of:
Component 1: 50 to 300 g/L
Component 2: 600 to 900 g/L
Component 3: 0 to 50 g/L
Component 4: 50 to 150 g/L
   wherein the amounts of components 1 to 4 and, optionally, further additives are selected to add up to 1 Liter.

Preferably the different components 1 to 4 are present in the amounts of:
Component 1: 50 to 300 g/L
Component 2: 500 to 850 g/L
Component 3: 0 to 30 g/L
Component 4: 50 to 150 g/L
   wherein the amounts of components 1 to 4 and, optionally, further additives are selected to add up to 1 Liter.

More preferred, the different components 1 to 4 are present in the amounts of:
Component 1: 50 to 300 g/L
Component 2: 450 to 830 g/L
Component 3: 0 to 25 g/L
Component 4: 65 to 135 g/L
   wherein the amounts of components 1 to 4 and, optionally, further additives are selected to add up to 1 Liter.

Even more preferred amounts of components 1 to 4 and 5, if present, are disclosed in the Examples 6 to 10 described herein.

To prepare such formulations, biomass comprising spores of fusaricidin producing bacteria, preferably in dried form, and preferably produced via spray drying, is dispersed in a liquid continuous phase or part of it (as premix). For dispersion and deagglomeration of spore aggregates, a high-shear mixing equipment (e.g. a Siefer colloid mill, or a Silverson, Ultraturrax or Polytron mixer) can be used. Preferably the spore aggregates have a final average particle size of 1 to 25 μm, preferably 1 to 10 μm, more preferably 1 to 8 μm (determined with a light scattering method in liquid dispersion according to CIPAC method 187). Category 1 additives, category 2 additives, category 3 additives and auxiliaries can be added depending on their shear stability before or after introduction of the biomass.

Accordingly, another aspect of the present invention are processes for making formulations. Accordingly, all components and their individual amounts described herein as being suitable for solid or liquid formulations are equally suitable for processes for making such formulations.

One process is a process for making solid formulations normally comprise the following steps:
a) providing biomass comprising spores of fusaricidin producing bacteria
b) adding at least one category 1 additive, category 2 additive, category 3 additive or auxiliaries to the selected final amount of the solid formulation
c) mixing to form a homogenous slurry or dough and
d) drying the solid formulation preferably by extrusion and post-drying or by spray-drying.
Wherein the steps a) and b) can be performed in reverse order and/or repeated together with step c) till all components of the solid formulation have been provided.

Further aspects are processes for making solid formulation, preferably a wettable powder (WP) formulation, comprising:
a) 5.0% w/w to 50% w/w biomass comprising spores of fusaricidin producing bacteria,
b) 5.0% w/w to 50% w/w of a category 1 additive,
c) 0% w/w to 20% w/w of a category 2 additive,
d) 0% w/w to 2% w/w of a category 3 additive, and
e) 30% w/w to 50% w/w of auxiliaries.
   wherein the total amounts of a), b), c), d), and e) are selected to result in a total amount of
100% w/w, comprising the following steps:
A. providing biomass comprising spores of fusaricidin producing bacteria,
B. adding at least one category 1 additive,
C. optionally adding at least one category 2 additive,
D. optionally adding at least one category 3 additive,
E. optionally adding auxiliaries,
F. drying the biomass containing slurry by spray-drying, vacuum-drum drying, fluidized-bed
drying or spray-granulation,
Wherein steps C), D), E) and F) can be carried out at any time during the process.

A further process is a process for making liquid formulations comprising the following steps:
a) providing biomass comprising spores of fusaricidin producing bacteria in a highly concentrated or dry manner
b) adding at least one category 1 additive, category 2 additive, category 3 additive and auxiliary,
c) adding water and/or one or more water-soluble organic liquids,
d) mixing to form a homogenous liquid formulation.

Wherein the steps a) and b) can be performed in reverse order and/or repeated together with step c) till all components of the solid formulation have been provided.

Another process is a process for making liquid formulations comprising the following steps:
a) providing a broth concentrate comprising spores of fusaricidin producing bacteria
b) dispersing the broth concentrate in a continuous phase, wherein the continuous phase comprises
   a. at least one category 1 additive
   b. optionally at least one category 2 additive
   c. optionally at least one stabilizer
   d. optionally further additives.

The stabilizer is preferably at least one soluble salts like calcium nitrate, calcium chloride, sodium chloride, potassium chloride, small organic acids like sorbic acid, lactic acid, benzoic acid and their sodium or potassium salts, or acids (citric acid, sulfuric acid, acetic acid, lactic acid) or buffers to provide slightly acidic pH for effectiveness of small organic acids.

Also included are processes for making a suspension concentrate (SC) formulation comprising:
a) providing biomass comprising spores of fusaricidin producing bacteria,
b) dispersing said biomass by shear or mechanical energy input, preferably in a rotor-stator colloid mill or a bead mill, in a continuous phase comprising,
c) adding at least one category 1 additive,
d) optionally adding at least one category 2 additive,
e) optionally adding at least one category 3 additive,
f) optionally adding auxiliaries,
   wherein steps c), d), e) and f) can be carried out at any time during the process.

Another aspect of the present invention are methods of combating phytopathogenic fungi, including phytopathogenic oomycetes, using formulations according to the invention to prepare water based or oil based spraying liquids, which are then applied to the plants.

The user applies the spraying liquids according to the invention usually from a predosage device, a knapsack sprayer, a spray tank, a spray plane, or an irrigation system. Usually, the agrochemical spraying liquid is made up with water, buffer, and/or further auxiliaries to the desired application concentration. Usually, 20 to 2000 liters, preferably between 50, 100, 200, 300, 400, 500, 600, 700, 800, 900 to 1000 liters, of the spraying liquid is applied per hectare of agricultural useful area. To enable even distribution of the spraying liquid to all parts of the plants, a higher volume of spraying liquid is preferred.

Phytopathogenic fungi combated with the spraying liquids of the invention are Albugo spp. (white rust) on ornamentals, vegetables (e. g. A. candida) and sunflowers (e. g. A. tragopogonis); Alternaria spp. (Alternaria leaf spot) on vegetables (e.g. A. dauci or A. porri), oilseed rape (A. brassicicola or brassicae), sugar beets (A. tenuis), fruits (e.g. A. grandis), rice, soybeans, potatoes and tomatoes (e. g. A. solani, A. grandis or A. alternata), tomatoes (e. g. A. solani or A. alternata) and wheat (e.g. A. triticina); Aphanomyces spp. on sugar beets and vegetables; Ascochyta spp. on cereals and vegetables, e. g. A. tritici (anthracnose) on wheat and A. hordei on barley; Aureobasidium zeae (syn. Kapatiella zeae) on corn; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages); B. squamosa or B. allii on onion family), oilseed rape, ornamentals (e.g. B. eliptica), vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladobotryum (syn. Dactylium) spp. (e.g. C. mycophilum (formerly Dactylium dendroides, teleomorph: Nectria albertinii, Nectria rosella syn. Hypomyces rosellus) on mushrooms; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum), soybeans (e. g. C. truncatum or C. gloeosporioides), vegetables (e.g. C. lagenarium or C. capsici), fruits (e.g. C. acutatum), coffee (e.g. C. coffeanum or C. kahawae) and C. gloeosporioides on various crops; Corticium spp., e. g. C. sasakii (sheath blight) on rice; Corynespora cassiicola (leaf spots) on soybeans, cotton and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, oilseed rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals, potatoes and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (syn. Monilia spp.: bloom and twig blight, brown rot) on stone fruits and other rosaceous plants; Mycosphaerella spp. on cereals, bananas, soft fruits and ground nuts, such as e. g. M. graminicola (anamorph: Zymoseptoria tritici formerly Septoria tritici: Septoria blotch) on wheat or M. fijiensis (syn. Pseudocercospora fijiensis: black Sigatoka disease) and M. musicola on bananas, M. arachidicola (syn. M. arachidis or Cercospora arachidis), M. berkeleyi on peanuts, M. pisi on peas and M. brassiciola on brassicas; Peronospora spp. (downy mildew) on cabbage (e. g. P. brassicae), oilseed rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (syn. Leptosphaeria biglobosa and L. maculans: root and stem rot) on oilseed rape and cabbage, P. betae (root rot, leaf spot and damping-off) on sugar beets and P. zeae-maydis (syn. Phyllostica zeae) on corn; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsid), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, oilseed rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits (e. g. P. leucotricha on apples) and curcurbits (P. xanthic); Polymyxa spp., e. g. on cereals, such as barley and wheat (P. graminis) and sugar beets (P. betae) and thereby transmitted viral diseases; Pseudocercosporella herpotrichoides (syn. Oculimacula yallundae, O. acuformis: eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenopeziza spp., e.g. P. brassicae on oilseed rape; Pyrenophora (anamorph: Drechslera) tritici-repentis (tan spot) on wheat or P. teres (net blotch) on barley; Pyricularia spp., e. g. P. oryzae (teleomorph: Magnaporthe grisea: rice blast) on rice and P. grisea on turf and cereals; Pythium spp. (damping-off) on turf, rice, corn, wheat, cotton, oilseed rape, sunflowers, soybeans, sugar beets, vegetables and various other plants (e. g. P. ultimum or P. aphanidermatum) and P. oligandrum on mushrooms; Ramularia spp., e. g. R. collo-cygni (Ramularia leaf spots, Physiological leaf spots) on barley, R. areola (teleomorph: Mycosphaerella areola) on cotton and R. beticola on sugar beets; Rhizoctonia spp. on cotton, rice, potatoes, turf, corn, oilseed rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis and R. commune (scald) on barley, rye and triticale; Sarocladium oryzae and S. attenuatum (sheath rot) on rice; Sclerotinia spp. (stem rot or white mold) on vegetables (S. minor and S. sclerotiorum) and field crops, such as oilseed rape, sunflowers (e. g. S. sclerotiorum) and soybeans, S. rolfsii (syn. Athelia rolfsii) on soybeans, peanut, vegetables, corn, cereals and ornamentals; Septoria spp. on various plants, e. g. S. glycines (brown spot) on soybeans, S. tritici (syn. Zymoseptoria tritici, Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setosphaeria spp. (leaf blight) on corn (e. g. S. turcicum, syn. Helminthosporium turcicum) and turf; Sphacelotheca spp. (smut) on corn, (e. g. S. reiliana, syn. Ustilago reiliana: head smut), sorghum and sugar cane; Sphaerotheca fuliginea (syn. Podosphaera xanthii: powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum, syn. Septoria nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Trichoderma harzianum on mushrooms; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli), sugar beets (e. g. U. betae or U. beticola) and on pulses (e.g. U. vignae, U. pisi, U. viciae-fabae and U. fabae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. longisporum on oilseed rape, V. dahliae on strawberries, oilseed rape, potatoes and tomatoes, and V. fungicola on mushrooms; Zymoseptoria tritici on cereals.

Preferred phytopathogenic fungi are: Alternaria spp., Botrytis spp., Phytophthora spp, Pythium spp, Plasmopara spp, Sclerotinia spp., Fusarium spp.,

Preferred phytopathogenic fungi species are: A. solani and A. alternata, Botrytis cinerea, Phytophthora infestans, and Sclerotinia sclerotiorum;

Plants to be treated with the spraying liquids of the invention include all plants which are attacked or are in danger of being attacked by one or more of the phytopathogenic fungi listed above. Preferred plants include durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, pepper, beans or squashes.

Another aspect of the present invention are methods of treating seeds using formulations according to the invention or prepared according to the invention.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The invention also comprises seeds coated with or containing the active formulation. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seeds are for example, but not excluding others, seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugar beet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The invention therefore also relates to seed to which a formulation of the invention has been applied. The amount of the active ingredients of formulations the invention will in general vary from 0.1 g to 10 kg per 100 kg of seed, preferably from 1 g to 5 kg per 100 kg of seed, in particular from 1 g to 1000 g per 100 kg of seed.

When the spraying liquids are applied as foliar treatment or to the soil, the application rates usually range from about 1×10⁶ to 5×10¹⁵ (or more) CFU/ha, preferably from about 1×10⁷ to about 1×10¹³ CFU/ha, even more preferably from 1×10⁹ to 5×10¹² CFU/ha. In one embodiment the application rates range from about 2×10⁹ to 4×10⁹ CFU/ha.

When the formulations of the invention are employed in seed treatment, the application rates with respect to plant propagation material usually range from about 1×10¹ to 1×10¹² (or more) CFU/seed, preferably from about 1×10³ to about 1×10¹⁰ CFU/seed, and even more preferably from about 1×10³ to about 1×10⁶ CFU/seed. Alternatively, the application rates with respect to plant propagation material preferably range from about 1×10⁷ to 1×10¹⁶ (or more) CFU per 100 kg of seed, preferably from 1×10⁹ to about 1×10¹⁵ CFU per 100 kg of seed, even more preferably from 1×10¹¹ to about 1×10¹⁵ CFU per 100 kg of seed.

Formulations according to the invention are easy and economical to make and are environmentally friendly and non-toxic.

Formulations according to the invention can be prepared with small average particle sizes, low viscosity and with well dispersed bacterium spores.

Formulations according to the invention are very stable and have a low tendency to undergo sedimentation or to form agglomerates of the bacterium spores. Formulations according to the invention show little increase of viscosity over time.

Formulations according to the invention are stable against decomposition and have a low tendency to develop discoloration or undesired odor and are easy to handle and to spray.

Formulations according to the invention can be distributed homogeneously and evenly on the target and show excellent biological performance.

The embodiments listed below represent preferred exemplary embodiments of the invention but should not be understood to limit the invention. The amounts of category 1 additives, category 2 additives and, if present, category 3 additives, as well as the particular combinations of category 1 additives, category 2 additives and, if present, category 3 additives, disclosed in Examples 6 to 10 are especially preferred.

Embodiment 1: Spraying liquid suitable for spraying plants, comprising
d. biomass comprising spores of fusaricidin producing bacteria, in an amount of 50 g per hectare to 2000 g per hectare,
e. at least one category 1 additive or at least one category 2 additive or a mixture of at least one category 1 additive and at least one category 2 additive, in a total amount of 20 g per hectare to 2000 g per hectare, and
f. a total amount of less than 81 g per hectare category 3 additives,
   wherein the at least one category 1 additive is selected from at least one group of additives 1a) to 1f):
1a) poloxamers, polyethylene glycol (A)—polypropylene glycol (B) block copolymers of the A-B-A or B-A-B type, comprising polyethylene glycol-polypropylene glycol-polyethylene glycol block polymers with a molar mass of approximately 950 to 6500 g/mol and a polyethylene oxide content of 10% to 50% w/w;
1b) polyvinylpyrolidines, polyvinylacetates, and statistical and block copolymers of polyvinyl pyrrolidone and polyvinyl acetate;
1c) polyoxyethylene sorbitol fatty acid esters (“polysorbates”), having a fatty acid chain with a length of 10 to 20 carbon atoms, preferably 12 to 18 carbon atoms, most preferably dodecanoic acid and oleic acid and linoleic acid, from 10 to 150 oxyethylene units, preferably 20 to 80 oxyethylene units, most preferred 20 to 40 oxyethylene units;
1d) fatty acid glycerol esters including fatty acid mono- and diesters of glycerol and polyglycerol, glycerolmonooleate, glyceroldioleate;
1e) alcohol alcoxylates, preferably C10-18 alcohols, more preferably C10-C13 alcohols, most preferably C10 alcohols, from 5 to 80 oxyethylene units, more preferably from 5 to 40 oxyethylene units, most preferred from 7 to 14 oxyethylene units;
1f) alkyl glucosides having an alkyl chain length C5-C15 and 1 to 10 glucoside units, and
   wherein the at least one category 2 additive is selected from at least one group of additives 2a) to 2c)
2a) polyethylene glycol-grafted polyacrylates, and salts thereof, preferably sodium salts thereof;
2b) ethoxylated polyethyleneimines;
2c) sodium lauryl ether sulfate, preferably sodium dodecylpoly(oxyethylene)sulfates with 1 to 50 oxyethylene units, preferably with 1 to 7 oxyethylene units, most preferred with 1 to 4 oxyethylene units,
   wherein the category 3 additive belongs to a group of additives 3a) to 3g)
3a) lignosulfonates;
3b) alkylbenzene sulfonates and salts thereof;
3c) alkylnaphthalene sulfonates and salts thereof;
3e) napthalene sulfonic acid co-condensates;
3f) alkyl sulfonates and salts thereof;
3g) fatty alcohol sulfates and salts thereof, preferably sodium lauryl sulfate.

Embodiment 2: A spraying liquid according to embodiment 1, wherein the at least one category 1 additive of the groups 1a) to 1f) is selected from:
for group 1a): poloxamers;
for group 1b) statistical copolymers of polyvinyl pyrrolidone and polyvinyl acetate;
for group 1c) polyoxyethylene sorbitol oleic acid esters with 20 to 40 oxyethylene units;
for group 1d) fatty acid mono- and diesters of glycerol and polyglycerol
for group 1e) alcohol alcoxylates with 5 to 80 oxyethylene units;
for group 1f) alkyl polyglucosides having an alkyl chain length C8-C10 and mono- and diglucosides with an overall polymerization degree of 1.5.

Embodiment 3: A spraying liquid according to embodiment 1 or 2, wherein the at least one category 1 additive of the groups 1a) to 1f) is selected from:
for group 1a) from polyethylene—polypropylene glycol block polymer with 10% ethyleneoxide, 850 g/mol, polyethylene—polypropylene glycol block polymer with 40% ethyleneoxide, 3250 g/mol, polyethylene—polypropylene glycol block polymer with 20% ethyleneoxide, 3250 g/mol, or polyethylene—polypropylene glycol block polymer with 50% ethyleneoxide, 1750 g/mol;
for group 1b) polyvinylpyrrolidone with an average molar mass of maximally 50 000 g/mol, or polyvinylpyrrolidone-polyvinylacetate statistical copolymer;
for group 1c) polyoxyethylene (40) sorbitol hexaoleate, sorbitol ethoxylate esters, poly oxyethylene (20) sorbitan monolaurate, poly(oxyethylene) (20) sorbitan monooleate, sorbitan monooleate or sorbitol ethoxylate ester of polymerized fatty acid;
for group 1d) mixtures of polyglycerolesters and fatty acid esters, or glycerolmon/diooleat;
for group 1e) alkyl polyethylene glycol ether from C10-Guerbet alcohol, HLB 13, alkyl polyethylene glycol ether from C10-Guerbet alcohol, HLB 16, polyoxyethylene (23) lauryl ether or alkyl polyethylene glycol ether from C16 C18 fatty alcohol, HLB 18.5;
for group 1f) alkyl polyglucoside.

Embodiment 4: A spraying liquid according to embodiment 3, wherein the at least one category 1 additive is
a. a mixture of polyglycerolesters and fatty acid esters,
b. a polyethylene—polypropylene glycol block polymer with 10% ethyleneoxide, 850 g/mol,
c. a polyethylene—polypropylene glycol block polymer with 20% ethyleneoxide, 3250 g/mol,
d. a polyethylene—polypropylene glycol block polymer with 40% ethyleneoxide, 3250 g/mol,
e. a polyethylene—polypropylene glycol block polymer with 50% ethyleneoxide, 1750 g/mol,
f. a polyvinylpyrrolidone with an average molar mass of maximally 50 000 g/mol,
g. a poly(oxyethylene) (20) sorbitan monooleate,
h. a sorbitan monooleate,
i. an alkyl polyglucoside, or
g. a mixture of at least two category 1 additives selected from a., b., c., d. e., f., g., h. or i.

Embodiment 5: A spraying liquid according to embodiment 3, wherein the at least one category 2 additive of the groups 2a) to 2c) is selected from:
for group 2a) methyl-methacrylate graft copolymer,
for group 2b) ethoxylated cationic polyethyleneimine,
for group 2c) sodium lauryl ether sulfate.

Embodiment 6: A spraying liquid according to embodiments 4 or 5, wherein the at least one category 1 additive is selected from:
a. a mixture of polyglycerolesters and fatty acid esters,
b. a polyethylene—polypropylene glycol block polymer with 10% ethyleneoxide, 850 g/mol,
c. a polyethylene—polypropylene glycol block polymer with 20% ethyleneoxide, 3250 g/mol,
d. a polyethylene—polypropylene glycol block polymer with 40% ethyleneoxide, 3250 g/mol,
e. or a polyethylene—polypropylene glycol block polymer with 50% ethyleneoxide, 1750 g/mol, or
f. an alkyl polyglucoside,
g. a polyvinylpyrrolidone with an average molar mass of maximally 50 000 g/mol, and the category 2 additive is selected from
h. a methyl-methacrylate graft copolymer,
i. ethoxylated cationic polyethyleneimine or
j. sodium laurylethersulfate,
   or the category 1 and category 2 additives are a mixture of at least a one category 1 additive of a., b., c., d., e, f, or g. and at least one category 2 additive of h., i. or j.

Embodiment 7: A spraying liquid according to any one of embodiments 4 to 6, comprising:
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives,
b. at least one polyethylene—polypropylene glycol block polymer with 50% ethyleneoxide, 1750 g/mol and at least one mixture of polyglycerolesters and fatty acid esters as category 1 additives,
c. at least one polyethylene—polypropylene glycol block polymer with 50% ethyleneoxide, 1750 g/mol and at least one polyethylene—polypropylene glycol block polymer with 40% ethyleneoxide, 3250 g/mol as category 1 additives,
d. at least one poly(oxyethylene) (20) sorbitan monooleate and at least one sorbitan monooleate, or
e. at least one polyethylene—polypropylene glycol block polymer with 10% ethyleneoxide, 850 g/mol and at least one a polyvinylpyrrolidone with an average molar mass of maximally 50 000 g/mol as category 1 additives.

Embodiment 8: A spraying liquid according to embodiment 6 or 7, comprising:
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives and at least one sodium laurylethersulfate as category 2 additive
b. at least one polyethylene—polypropylene glycol block polymer with 20% ethyleneoxide, 3250 g/mol as category 1 additive and at least one methyl-methacrylate graft copolymer as category 2 additive,
c. at least one polyethylene—polypropylene glycol block polymer with 50% ethyleneoxide, 1750 g/mol as category 1 additive and at least one methyl-methacrylate graft copolymer as category 2 additive,
d. at least one polyethylene—polypropylene glycol block polymer with 50% ethyleneoxide, 1750 g/mol and at least one mixture of polyglycerolesters and fatty acid esters as category 1 additives and at least one methyl-methacrylate graft copolymer as category 2 additive,
e. at least one polyethylene—polypropylene glycol block polymer with 50% ethyleneoxide, 1750 g/mol and at least one polyethylene—polypropylene glycol block polymer with 40% ethyleneoxide, 3250 g/mol as category 1 additives and at least one methyl-methacrylate graft copolymer as category 2 additive,
f. at least one polyethylene—polypropylene glycol block polymer with 40% ethyleneoxide, 3250 g/mol as category 1 additive and at least one methyl-methacrylate graft copolymer as category 2 additive, or
g. at least one alkyl polyglucoside as category 1 additive and at least one methyl-methacrylate graft copolymer as category 2 additive.

Embodiment 9: A spraying liquid according to any one of embodiments 4 to 8, comprising:
a. a polyethylene—polypropylene glycol block polymer with 50% ethyleneoxide, 1750 g/mol and a polyethylene—polypropylene glycol block polymer with 20% ethyleneoxide, 3250 g/mol as category 1 additives, or
b. a polyethylene—polypropylene glycol block polymer with 50% ethyleneoxide, 1750 g/mol and a mixture of polyglycerolesters and fatty acid esters as category 1 additives,
c. a polyethylene—polypropylene glycol block polymer with 50% ethyleneoxide, 1750 g/mol and a polyethylene—polypropylene glycol block polymer with 40% ethyleneoxide, 3250 g/mol as category 1 additives.

Embodiment 10: A spraying liquid according to any one of embodiments 6 to 8, comprising:
a. polyethylene—polypropylene glycol block polymer with 50% ethyleneoxide, 1750 g/mol and a polyethylene—polypropylene glycol block polymer with 20% ethyleneoxide, 3250 g/mol as category 1 additives and a sodium laurylethersulfate as category 2 additive,
b. a polyethylene—polypropylene glycol block polymer with 20% ethyleneoxide, 3250 g/mol as category 1 additive and a methyl-methacrylate graft copolymer as category 2 additive,
c. a polyethylene—polypropylene glycol block polymer with 50% ethyleneoxide, 1750 g/mol as category 1 additive and a methyl-methacrylate graft copolymer as category 2 additive,
d. a polyethylene—polypropylene glycol block polymer with 50% ethyleneoxide, 1750 g/mol and a mixture of polyglycerolesters and fatty acid esters as category 1 additives and a methyl-methacrylate graft copolymer as category 2 additive,
e. a polyethylene—polypropylene glycol block polymer with 50% ethyleneoxide, 1750 g/mol and a polyethylene—polypropylene glycol block polymer with 40% ethyleneoxide, 3250 g/mol as category 1 additives and a methyl-methacrylate graft copolymer as category 2 additive,
f. a polyethylene—polypropylene glycol block polymer with 40% ethyleneoxide, 3250) as category 1 additive and a methyl-methacrylate graft copolymer as category 2 additive, or
g. an alkyl polyglucoside as category 1 additive and a methyl-methacrylate graft copolymer as category 2 additive.

Embodiment 11: A spraying liquid according to any one of embodiments 1 to 10, additionally comprising a category 3 additive, in a total amount of more than 0 g per hectare to 80 g per hectare, and being selected from at least one group of additives 3a) to 3g)
3a) lignosulfonates;
3b) alkylbenzene sulfonates and salts thereof;
3c) alkylnaphthalin sulfonates and salts thereof;
3e) napthalene sulfonic acid co-condensates;
3f) alkyl sulfonates and salts thereof;
3g) fatty alcohol sulfates and salts thereof, preferably sodium lauryl sulfate.

Embodiment 12: Kit of at least two parts to prepare a spraying liquid according to any one of the embodiments 1 to 11, wherein the biomass comprising spores of fusaricidin producing bacteria are provided in a first concentrated form and at least one of the category 1 additive and/or category 2 additive and/or category 3 additive are provided in at least one further concentrated forms and wherein the relative amounts of the biomass comprising spores of fusaricidin producing bacteria and the relative amounts the category 1 additive and/or the category 2 additive and/or the category 3 additive in the kit are adapted to provide the amounts described in any one of the embodiments 1 to 11.

Embodiment 13: Kit of parts according to embodiment 12, wherein the spraying liquid is prepared to be used in a method to control phytopathogenic fungi.

Embodiment 14: Method to control phytopathogenic fungi wherein a spraying liquid according to any one of embodiments 1 to 11 is sprayed on plants.

Embodiment 15: Method to control phytopathogenic fungi according to embodiment 14, wherein the spraying liquid is sprayed in an amount of about 100 liter to about 1000 liter per hectare and the spraying liquid comprises biomass comprising spores of fusaricidin producing bacteria in an amount of 0.005% w/w to 2% w/w and the category 1 additive and/or the category 2 additive in a total amount of 0.005% w/w to 2% w/w.

Embodiment 16: Method to control phytopathogenic fungi according to embodiment 15, wherein the spraying liquid comprises a category 3 additive in an amount of 0.0001% w/w to 0.08% w/w.

Embodiment 17: Method to control phytopathogenic fungi according to any one of embodiments 14 to 17, wherein the plants are selected from maize, small-grain cereals, cotton, canola, sunflowers, tobacco, peanuts, vegetables, grapevines, leafy vegetables, root & tuber vegetables, onions, strawberries, blueberries, strawberry, black currant, white currant, red currant, blackberry, raspberry, tomato, pepper, chilli, cucumber, aubergine, melons, watermelons, cucumbers, tomatoes, peppers & aubergines, herbs, turf and ornamentals.

Embodiment 18: Method to control phytopathogenic fungi according to any one of embodiments 14 to 17, wherein the phytopathogenic fungi is Alternaria spp., Botrytis cinerea or Phytophthora infestans.

Embodiment 19: A solid or liquid formulation, preferably a wettable powder (WC) or suspension concentrate (SC) formulation, comprising:
a. 5% w/w to 50% w/w biomass comprising spores of fusaricidin producing bacteria,
b. 5% w/w to 50% w/w of at least one category 1 additive or at least one category 2 additive or a mixture of at least one category 1 additive or at least one category 2 additive, and
c. less than 2% w/w of category 3 additives,
   wherein the at least one category 1 additive is selected from at least one group of additives 1a) to 1f):
1a) poloxamers, polyethylene glycol, polypropylene glycol or polyethylene glycol block polymers with a molar mass of approximately 950 to 6500 g/mol and a polyethylene oxide content of 10% to 50% w/w;
1b) polyvinylpyrolidines, polyvinylacetates and statistical and block copolymers of polyvinyl pyrrolidone and polyvinyl acetate, all with a molar mass of approximately 9000 to 70000 g/mol;
1c) polyoxyethylene sorbitol fatty acid esters (“polysorbates”), having a fatty acid chain with a length of 10 to 20 carbon atoms, preferably 12 to 18 carbon atoms, most preferably dodecanoic acid and oleic acid and linoleic acid, from 10 to 150 oxyethylene units, preferably 20 to 80 oxyethylene units, most preferred 20 to 40 oxyethylene units;
1d) fatty acid glycerol esters, fatty acid mono- and diesters of glycerol and polyglycerol, glycerolmonooleate, glyceroldioleate, polyglycerol fatty acid ester;
1e) alcohol alcoxylates, preferably C10-18 alcohols, more preferably C10-C13 alcohols, most preferably C10 alcohols, from 5 to 80 oxyethylene units, more preferably from 5 to 40 oxyethylene units, most preferred from 7 to 14 oxyethylene units;
1f) alkyl glucosides having an alkyl chain length C5-C15 and 1 to 10 glucoside units, and the at least one category 2 additive is selected from at least one group of additives 2a) to 2c)
2a) polyethylene glycol-grafted polyacrylates, and salts thereof, preferably sodium salts thereof;
2b) ethoxylated polyethyleneimines;
2c) sodium lauryl ether sulfate, preferably sodium dodecylpoly(oxyethylene)sulfates with 1 to 50 oxyethylene units, preferably with 1 to 7 oxyethylene units, most preferred with 1 to 4 oxyethylene units,
and the category 3 additives are selected from the groups of additives 3a) to 3g)
3a) lignosulfonates;
3b) alkylbenzene sulfonates and salts thereof;
3c) alkylnaphthalene sulfonates and salts thereof;
3e) apthalene sulfonic acid co-condensates;
3f) alkyl sulfonates and salts thereof;
3g) fatty alcohol sulfates and salts thereof, preferably sodium lauryl sulfate.

Embodiment 20: A solid or liquid formulation, preferably a wettable powder (WP) or suspension concentrate (SC) formulation, according to embodiment 19, wherein the at least one category 1 additive of the groups 1a) to 1f) is selected from:
for group 1a): poloxamers;
for group 1b) statistical copolymers of polyvinyl pyrrolidone and polivinyl acetate;
for group 1c) polyoxyethylene sorbitol oleic acid esters with 20 to 40 oxyethylene units
for group 1d) fatty acid mono- and diesters of glycerol and polyglycerol
for group 1e) alcohol alcoxylates with 5 to 80 oxyethylene units,
for group 1f) alkyl polyglucosides having an alkyl chain length C8-C10 and mono- and diglucosides with an overall polymerization degree of 1.5.

Embodiment 21: A solid or liquid formulation, preferably a wettable powder (WP) or suspension concentrate (SC) formulation, according to embodiment 19 or 20, wherein the at least one category 1 additive of the groups 1a) to 1f) is selected from:
for group 1a) from polyethylene—polypropylene glycol block polymer with 10% ethyleneoxide, 850 g/mol, polyethylene—polypropylene glycol block polymer with 40% ethyleneoxide, 3250 g/mol, polyethylene—polypropylene glycol block polymer with 20% ethyleneoxide, 3250 g/mol or polyethylene—polypropylene glycol block polymer with 50% ethyleneoxide, 1750 g/mol;
for group 1b) polyvinylpyrrolidone with an average molar mass of maximally 50 000 g/mol, or polyvinylpyrrolidone-polyvinylacetate statistical copolymer;
for group 1c) polyoxyethylene (40) sorbitol hexaoleate, sorbitol ethoxylate esters, poly oxyethylene (20) sorbitan monolaurate, poly(oxyethylene) (20) sorbitan monooleate, sorbitan monooleate or sorbitol ethoxylate ester of polymerized fatty acid;
for group 1d) mixtures of polyglycerolesters and fatty acid esters or glycerolmon/diooleat;
for group 1e) alkyl polyethylene glycol ether from C10-Guerbet alcohol, HLB 13, alkyl polyethylene glycol ether from C10-Guerbet alcohol, HLB 16, polyoxyethylene (23) lauryl ether or alkyl polyethylene glycol ether from C16 C18 fatty alcohol, HLB 18.5;
for group 1f) alkyl polyglucoside.

Embodiment 22: A solid or liquid formulation, preferably a wettable powder (WP) or suspension concentrate (SC) formulation, according to any one of embodiments 19 to 21, wherein the at least one category 1 additive is
a. at least one mixture of polyglycerolesters and fatty acid esters,
b. at least one polyethylene—polypropylene glycol block polymer with 10% ethyleneoxide, 850 g/mol,
c. at least one polyethylene—polypropylene glycol block polymer with 20% ethyleneoxide, 3250 g/mol,
d. at least one polyethylene—polypropylene glycol block polymer with 40% ethyleneoxide, 3250 g/mol,
e. at least one polyethylene—polypropylene glycol block polymer with 50% ethyleneoxide, 1750 g/mol,
f. at least one polyvinylpyrrolidone with an average molar mass of maximally 50 000 g/mol,
g. at least one alkyl polyglucoside, or
h. a mixture of at least two category 1 additives selected from a., b., c., d., e., f., or g.

Embodiment 23: A solid or liquid formulation, preferably a wettable powder (WP) or suspension concentrate (SC) formulation, according to any one of embodiments 19 to 22, wherein the at least one category 2 additive of the groups 2a) to 2c) is selected from:
for group 2a) methyl-methacrylate graft copolymer,
for group 2b) ethoxylated cationic polyethyleneimine, or
for group 2c) sodium lauryl ether sulfate.

Embodiment 24: A solid or liquid formulation, preferably a wettable powder (WP) or suspension concentrate (SC) formulation according to any one of embodiments 19 to 23, wherein the at least one category 1 additive is selected from:
a. at least one mixture of polyglycerolesters and fatty acid esters,
b. at least one polyethylene—polypropylene glycol block polymer with 10% ethyleneoxide, 850 g/mol,
c. at least one polyethylene—polypropylene glycol block polymer with 20% ethyleneoxide, 3250 g/mol,
d. at least one polyethylene—polypropylene glycol block polymer with 40% ethyleneoxide, 3250 g/mol,
e. at least one polyethylene—polypropylene glycol block polymer with 50% ethyleneoxide, 1750 g/mol,
f. at least one polyvinylpyrrolidone with an average molar mass of maximally 50 000 g/mol or
g. at least one alkyl polyglucoside,
and the category 2 additive is selected from
h. at least one methyl-methacrylate graft copolymer,
i. at least one ethoxylated cationic polyethyleneimine or
j. at least one sodium laurylethersulfate,
or the category 1 and category 2 additives are a mixture of at least one category 1 additive of a., b., c., d., e., f., or g. and at least one category 2 additive of h., i. or j.

Embodiment 25: A solid or liquid formulation, preferably a wettable powder (WP) or suspension concentrate (SC) formulation according to any one of embodiment 19 to 24, comprising:
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives,
b. at least one polyethylene—polypropylene glycol block polymer with 50% ethyleneoxide, 1750 g/mol and at least one mixture of polyglycerolesters and fatty acid esters as category 1 additives,
c. at least one polyethylene—polypropylene glycol block polymer with 50% ethyleneoxide, 1750 g/mol and at least one polyethylene—polypropylene glycol block polymer with 40% ethyleneoxide, 3250 g/mol as category 1 additives,
d. at least one poly(oxyethylene) (20) sorbitan monooleate and at least one sorbitan monooleate as category 1 additives, or
e. at least one polyethylene—polypropylene glycol block polymer with 10% ethyleneoxide, 850 g/mol and at least one polyvinylpyrrolidone with an average molar mass of maximally 50 000 g/mol as category 1 additives.

Embodiment 26: A solid or liquid formulation, preferably a wettable powder (WP) or suspension concentrate (SC) formulation according to any one of embodiments 19 to 25, comprising:
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives and at least one sodium laurylethersulfate as category 2 additive,
b. at least one polyethylene—polypropylene glycol block polymer with 20% ethyleneoxide, 3250 g/mol as category 1 additive and at least one methyl-methacrylate graft copolymer as category 2 additive,
c. at least one polyethylene—polypropylene glycol block polymer with 50% ethyleneoxide, 1750 g/mol as category 1 additive and at least one methyl-methacrylate graft copolymer as category 2 additive,
d. at least one polyethylene—polypropylene glycol block polymer with 50% ethyleneoxide, 1750 g/mol and at least one mixture of polyglycerolesters and fatty acid esters as category 1 additives and at least one methyl-methacrylate graft copolymer as category 2 additive,
e. at least one polyethylene—polypropylene glycol block polymer with 50% ethyleneoxide, 1750 g/mol and at least one polyethylene—polypropylene glycol block polymer with 40% ethyleneoxide, 3250 g/mol as category 1 additives and at least one methyl-methacrylate graft copolymer as category 2 additive,
f. at least one polyethylene—polypropylene glycol block polymer with 40% ethyleneoxide, 3250 g/mol as category 1 additive and at least one methyl-methacrylate graft copolymer as category 2 additive, or
g. at least one alkyl polyglucoside as category 1 additive and at least one methyl-methacrylate graft copolymer as category 2 additive.

Embodiment 27: A solid or liquid formulation, preferably a wettable powder (WP) or suspension concentrate (SC) formulation according to any one of embodiments 19 to 26, comprising:
a. a polyethylene—polypropylene glycol block polymer with 50% ethyleneoxide, 1750 g/mol and a polyethylene—polypropylene glycol block polymer with 20% ethyleneoxide, 3250 g/mol as category 1 additives, or
b. a polyethylene—polypropylene glycol block polymer with 50% ethyleneoxide, 1750 g/mol and a mixture of polyglycerolesters and fatty acid esters as category 1 additives,
c. a polyethylene—polypropylene glycol block polymer with 50% ethyleneoxide, 1750 g/mol and a polyethylene—polypropylene glycol block polymer with 40% ethyleneoxide, 3250 g/mol as category 1 additives, or
d. a polyethylene—polypropylene glycol block polymer with 10% ethyleneoxide, 850 g/mol and a polyvinylpyrrolidone with an average molar mass of maximally 50 000 g/mol.

Embodiment 28: A solid or liquid formulation, preferably a wettable powder (WP) or suspension concentrate (SC) formulation according to any one of embodiments 19 to 27, comprising:
a. polyethylene—polypropylene glycol block polymer with 50% ethyleneoxide, 1750 g/mol and a polyethylene—polypropylene glycol block polymer with 20% ethyleneoxide, 3250 g/mol as category 1 additives and a sodium laurylethersulfate as category 2 additive,
b. a polyethylene—polypropylene glycol block polymer with 20% ethyleneoxide, 3250 g/mol as category 1 additive and a methyl-methacrylate graft copolymer as category 2 additive,
c. a polyethylene—polypropylene glycol block polymer with 50% ethyleneoxide, 1750 g/mol as category 1 additive and a methyl-methacrylate graft copolymer as category 2 additive,
d. a polyethylene—polypropylene glycol block polymer with 50% ethyleneoxide, 1750 g/mol and a mixture of polyglycerolesters and fatty acid esters as category 1 additives and a methyl-methacrylate graft copolymer as category 2 additive,
e. a polyethylene—polypropylene glycol block polymer with 50% ethyleneoxide, 1750 g/mol and a polyethylene—polypropylene glycol block polymer with 40% ethyleneoxide, 3250 g/mol as category 1 additives and a methyl-methacrylate graft copolymer as category 2 additive,
f. a polyethylene—polypropylene glycol block polymer with 40% ethyleneoxide, 3250 g/mol as category 1 additive and a methyl-methacrylate graft copolymer as category 2 additive, or
g. an alkyl polyglucoside as category 1 additive and a methyl-methacrylate graft copolymer as category 2 additive.

Embodiment 29: A solid or liquid formulation, preferably a wettable powder (WP) or suspension concentrate (SC) formulation according to any one of embodiments 19 to 28, additionally comprising a category 3 additive, in a total amount of more than 0% w/w to 2% w/w, and being selected from at least one group of additives 3a) to 3g)
3a) lignosulfonates;
3b) alkylbenzene sulfonates and salts thereof;
3c) alkylnaphthalene sulfonates and salts thereof;
3e) napthalene sulfonic acid co-condensates;
3f) alkyl sulfonates and salts thereof;
3g) fatty alcohol sulfates and salts thereof, preferably sodium lauryl sulfate.

Embodiment 30: A solid or liquid formulation, preferably a wettable powder (WP) or suspension concentrate (SC) formulation according to any one of embodiment 19 to 29 comprising
a. 5% w/w to 50% w/w biomass comprising spores of fusaricidin producing bacteria,
b. 2% w/w to 50% w/w of a category 1 additive,
c. 0% w/w to 50% w/w of a category 2 additive,
d. 0 w/w to 2% w/w of a category 3 additive, and
e. 20% w/w to 75% w/w of auxiliaries.
   wherein the total amounts of a., b., c., d., and e. are selected to result in a total amount of 100% w/w.

Embodiment 31: A solid or liquid formulation, preferably a wettable powder (WP) or suspension concentrate (SC) formulation according to any one of embodiments 19 to 30 comprising
a. 10% w/w to 40% w/w biomass comprising spores of fusaricidin producing bacteria,
b. 10% w/w to 40% w/w of a category 1 additive,
c. 2.5% w/w to 20% w/w of a category 2 additive,
d. 0 w/w to 2% w/w of a category 3 additive, and
e. 20% w/w to 75% w/w of auxiliaries.
   wherein the total amounts of a., b., c., d., and e. are selected to result in a total amount of 100% w/w.

Embodiment 32: A solid or liquid formulation, preferably a wettable powder (WP) or suspension concentrate (SC) formulation according to any one of embodiments 19 to 31 comprising
a. 10% w/w to 35% w/w biomass comprising spores of fusaricidin producing bacteria,
b. 3% w/w to 40% w/w of a category 1 additive,
c. 0% w/w to 15% w/w of a category 2 additive,
d. 0 w/w to 2% w/w of a category 3 additive, and
e. 20% w/w to 75% w/w of auxiliaries.
   wherein the total amounts of a., b., c., d. and e. are selected to result in a total amount of 100% w/w.

Embodiment 33: A solid or liquid formulation, preferably a wettable powder (WP) or suspension concentrate (SC) formulation according to any one of embodiments 19 to 33 comprising
a. 15% w/w to 35% w/w biomass comprising spores of fusaricidin producing bacteria,
b. 3% w/w to 20% w/w of a category 1 additive,
c. 0% w/w to 7% w/w of a category 2 additive,
d. 0% w/w to 1.5% w/w of a category 3 additive, and
e. 20% w/w to 75% w/w of auxiliaries.
   wherein the total amounts of a., b., c., d. and e. are selected to result in a total amount of 100% w/w.

Embodiment 34: A solid or liquid formulation, preferably a wettable powder (WP) or suspension concentrate (SC) formulation according to any one of embodiments 19 to 33 comprising
a. 15% w/w to 40% w/w biomass comprising spores of fusaricidin producing bacteria,
b. 3% w/w to 40% w/w of a category 1 additive,
c. 2% w/w to 15% w/w of a category 2 additive,
d. 0% w/w of a category 3 additive, and
e. 30% w/w to 75% w/w of auxiliaries.
   wherein the total amounts of a., b., c., d. and e. are selected to result in a total amount of 100% w/w.

Embodiment 35: A solid formulation, preferably a wettable powder (WP) formulation, according to any one of embodiments 19 to 34, comprising 2% w/w to 50% w/w of a category 1 additive, wherein the category 1 additive is a combination of
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives,
b. at least one polyethylene—polypropylene glycol block polymer with 50% ethyleneoxide, 1750 g/mol and at least one mixture of polyglycerolesters and fatty acid esters as category 1 additives,
c. at least one poly(oxyethylene) (20) sorbitan monooleate and at least one sorbitan monooleate, or
d. at least one polyethylene—polypropylene glycol block polymer with 10% ethyleneoxide, 850 g/mol and at least one polyvinylpyrrolidone with an average molar mass of maximally 50 000 g/mol, as category 1 additives.

Embodiment 36: A solid formulation, preferably a wettable powder (WP) formulation, according embodiment 35, comprising 10% w/w to 50% w/w of a category 1 additive wherein the category 1 additive is a combination of
a. 5% w/w to 45% w/w of at least one polyethylene—polypropylene glycol block polymer with 50% ethyleneoxide, 1750 g/mol and
b. 5% w/w to 45% w/w of at least one polyethylene—polypropylene glycol block polymer with 20% ethyleneoxide, 3250 g/mol.

Embodiment 37: A solid formulation, preferably a wettable powder (WP) formulation, according embodiment 35, comprising 10% w/w to 50% w/w of a category 1 additive wherein the category 1 additive is a combination of
a. 9% w/w to 45% w/w of at least one polyethylene—polypropylene glycol block polymer with 50% ethyleneoxide, 1750 g/mol and 1% w/w to 10% w/w of at least one mixture of polyglycerolesters and fatty acid esters as category 1 additives.

Embodiment 38: A solid formulation, preferably a wettable powder (WP) formulation, according embodiment 35, comprising as category 1 additives:
a. 1.0% w/w to 20% w/w of at least one polyethylene—polypropylene glycol block polymer with 50% ethyleneoxide, 1750 g/mol and
b. 10% w/w to 19% w/w of at least one polyethylene—polypropylene glycol block polymer with 20% ethyleneoxide, 3250 g/mol.

Embodiment 39: A solid formulation, preferably a wettable powder (WP) formulation, according embodiment 35, comprising:
a. 7% w/w to 45% w/w of at least one polyethylene—polypropylene glycol block polymer with 50% ethyleneoxide, 1750 g/mol and 3% w/w to 10% w/w of at least one mixture of polyglycerolesters and fatty acid esters as category 1 additives.

Embodiment 40: A solid formulation, preferably a wettable powder (WP) formulation, according to any one of embodiments 36 to 38, comprising as category 1 additives:
a. 1.0% w/w to 20% w/w of at least one polyethylene—polypropylene glycol block polymer with 50% ethyleneoxide, 1750 g/mol and
b. 10% w/w to 19% w/w of at least one polyethylene—polypropylene glycol block polymer with 20% ethyleneoxide, 3250 g/mol and
   comprising as category 2 additive 0.5% w/w to 2% w/w of at least one sodium laurylethersulfate.

Embodiment 41: A solid formulation, preferably a wettable powder (WP) formulation, according to embodiment 40, comprising:
a. 1% w/w to 20% w/w of at least one polyethylene—polypropylene glycol block polymer with 50% ethyleneoxide, 1750 g/mol and
b. 10% w/w to 19% w/w of at least one polyethylene—polypropylene glycol block polymer with 20% ethyleneoxide, 3250 g/mol, and
c. 0.5% w/w to 14% w/w of at least one methyl-methacrylate graft copolymer.

Embodiment 42: A solid formulation, preferably a wettable powder (WP) formulation, according to embodiment 40, comprising:
a. 1% w/w to 20% w/w of at least one polyethylene—polypropylene glycol block polymer with 50% ethyleneoxide, 1750 g/mol and
b. 10% w/w to 19% w/w of at least one polyethylene—polypropylene glycol block polymer with 20% ethyleneoxide, 3250 g/mol, and
c. 0.8% w/w to 8% w/w of at least one methyl-methacrylate graft copolymer.

Embodiment 43: A solid formulation, preferably a wettable powder (WP) formulation, according to embodiment 35, comprising:
a. 7% w/w to 45% w/w of at least one polyethylene—polypropylene glycol block polymer with 50% ethyleneoxide, 1750 g/mol,
b. 3% w/w to 10% w/w of at least one mixture of polyglycerolesters and fatty acid esters as category 1 additives, and
c. 0.8% w/w to 14% w/w of at least one methyl-methacrylate graft copolymer.
2. A solid formulation, preferably a wettable powder (WP) formulation, according to claim 43, comprising:
a. 7% w/w to 45% w/w of at least one polyethylene—polypropylene glycol block polymer with 50% ethyleneoxide, 1750 g/mol,
b. 3% w/w to 10% w/w of at least one mixture of polyglycerolesters and fatty acid esters as category 1 additives, and
c. 0.8% w/w to 8% w/w of at least one methyl-methacrylate graft copolymer.

Embodiment 44: A solid formulation, preferably a wettable powder (WP) formulation, according to any one of embodiments 35 to 44, packed in vacuum sealed multifoil or mylar pouches, or pouches made of water-soluble polymers, e.g. polyvinyl alcohol pouches.

Embodiment 45: A liquid formulation, preferably suspension concentrate (SC) formulation, according to at least one of embodiments 19 to 33, comprising:
a. 2% w/w to 15% w/w of at least one category 1 additive selected from polyethylene—polypropylene glycol block polymer with 50% ethyleneoxide, 1750 g/mol, polyethylene—polypropylene glycol block polymer with 20% ethyleneoxide, 3250 g/mol, polyethylene—polypropylene glycol block polymer with 40% ethyleneoxide, 3250 g/mol and/or a mixture of polyglycerolesters and fatty acid esters, or a mixture of at least two of these, and
b. 1% w/w to 15% w/w of at least one category 2 additive selected from methyl-methacrylate graft copolymer.

Embodiment 46: A liquid formulation, preferably suspension concentrate (SC) formulation, according to at least one of embodiments 19 to 33 and 45, comprising:
a. 3% w/w to 10% w/w of a category 2 additive methyl-methacrylate graft copolymer and
b. at least one category 1 additive selected from the alternatives
   i. 3% w/w to 10% w/w polyethylene—polypropylene glycol block polymer with 20% ethyleneoxide, 3250 g/mol,
   ii. 2% w/w to 15% w/w of at least one category 1 additive selected from polyethylene—polypropylene glycol block polymer with 50% ethyleneoxide, 1750 g/mol,
   iii. 2% w/w to 10% w/w of at least one category 1 additive selected from polyethylene—polypropylene glycol block polymer with 50% ethyleneoxide, 1750 g/mol and 1% w/w to 5% w/w of a mixture of polyglycerolesters and fatty acid esters,
   iv. 3% w/w to 10% w/w of at least one category 1 additive selected from polyethylene—polypropylene glycol block polymer with 50% ethyleneoxide, 1750 g/mol and 3% w/w to 10% w/w of polyethylene—polypropylene glycol block polymer with 40% ethyleneoxide, 3250 g/mol.

Embodiment 47: A liquid formulation, preferably suspension concentrate (SC) formulation, according to at least one of embodiments 19 to 33 and 46 or 47, comprising
35% w/w to 45% w/w of glycerol, propane-1,2-diol (propylen glycol), butane-1,4-diol, butane-1,3-diol, pentane-1,5-diol, hexane-1,6-diol, 2-methyl-2,4-pentanediol (hexylene glycol), heptan-1,7-diol, 2-ethylhexan-1,3-diol, polyethylene glycol (PEG) with a mean molecular weight of 150 g/mol to 450 g/mol, polypropylene glycol (PPG) with a mean molecular weight of 200 to 500 g/mol, propylene glycol methyl ether, dipropylene glycol methyl ether, tripropylene glycol methyl ether, dipropylene glycol dimethyl ether, propylene glycol n-propyl ether, diethylene glycol monobutylether, 2-hydroxypropane-1,2,3-tricarboxylate (triethyl citrate), or N,N-dimethyl lactamide or a mixture of at least two of these.

Embodiment 48: A liquid formulation, preferably suspension concentrate (SC) formulation, according to at least one of embodiments 19 to 33 and 46 or 47, comprising 35% w/w to 45% w/w of polyethylene glycol (PEG), propane-1,2-diol, 2-methyl-2,4-pentanediol or triethyl citrate or a mixture of at least two of these.

Embodiment 49: A liquid formulation, preferably suspension concentrate (SC) formulation, according to at least one of embodiments 19 to 33 and 46, comprising:
a. 6% w/w to 15% w/w of the category 2 additive methyl-methacrylate graft copolymer and
b. 2% w/w to 8% w/w of the category 1 additive polyethylene—polypropylene glycol block polymer with 40% ethyleneoxide, 3250 g/mol.

Embodiment 50: A liquid formulation, preferably suspension concentrate (SC) formulation, according to at least one of embodiments 19 to 33 and 46, comprising:
a. 6% w/w to 15% w/w of the category 2 additive methyl-methacrylate graft copolymer and
b. 3% w/w to 8% w/w of the category 3 additive alkyl polyglucoside.

Embodiment 51: A liquid formulation, preferably suspension concentrate (SC) formulation, according to at least one of embodiment 19 to 33 and 46 or 47, comprising 70% w/w to 85% w/w of glycerol, propane-1,2-diol (propylen glycol), butane-1,4-diol, butane-1,3-diol, pentane-1,5-diol, hexane-1,6-diol, 2-methyl-2,4-pentanediol (hexylene glycol), polyethylene glycol (PEG) with a mean molecular weight of 150 g/mol to 450 g/mol, polypropylene glycol (PPG) with a mean molecular weight of 200 to 500 g/mol, propylene glycol methyl ether, dipropylene glycol methyl ether, tripropylene glycol methyl ether, dipropylene glycol dimethyl ether, propylene glycol n-propyl ether, diethylene glycol monobutylether, triethyl 2-hydroxypropane-1,2,3-tricarboxylate (triethyl citrate), tributyl 2-hydroxypropane-1,2,3-tricarboxylat (tributyl citrate), or N,N-dimethyl lactamide or a mixture of at least two of these.

Embodiment 52: A liquid formulation, preferably suspension concentrate (SC) formulation, according to embodiments 50 or 51, comprising 75% w/w to 85% w/w of polyethylene glycol (PEG), propane-1,2-diol, 2-methyl-2,4-pentanediol, triethyl citrate or tributyl citrate or a mixture of at least two of these, preferably polyethylene glycol (PEG), 2-methyl-2,4-pentanediol, triethyl citrate or tributyl citrate or a mixture of at least two of these, even more preferred polyethylene glycol (PEG), 2-methyl-2,4-pentanediol or triethyl citrate, or a mixture of at least two of these.

Embodiment 53: Method to control phytopathogenic fungi on plants comprising
a. providing at least one solid or liquid formulation, preferably a wettable powder (WP) or suspension concentrate (SC) formulation, according to any one of embodiments 19 to 52 in a total amount of 0.5 kg to 4 kg per hectare or a kit of parts according to embodiments 12 or 13,
b. adding water to a final amount of 100 liters to 1000 liters per hectare, and
c. spraying the resulting spraying liquid on plants.

Embodiment 54: Process for making solid formulation, preferably a wettable powder (WP) formulation, comprising:
a. 5.0% w/w to 50% w/w biomass comprising spores of fusaricidin producing bacteria,
b. 5.0% w/w to 50% w/w of a category 1 additive,
c. 0% w/w to 20% w/w of a category 2 additive,
d. 0 w/w to 2% w/w of a category 3 additive, and
e. 30% w/w to 50% w/w of auxiliaries.
   wherein the total amounts of a, b, c, d, and e are selected to result in a total amount of 100% w/w,
   comprising the following steps:
A. providing biomass comprising spores of fusaricidin producing bacteria,
B. dispersing said biomass in a continuous phase comprising,
C. adding at least one category 1 additive,
D. optionally adding at least one category 2 additive,
E. optionally adding at least one category 3 additive,
F. optionally adding auxiliaries,
G. drying the biomass containing slurry preferably by spray-drying, vacuum-drum drying, fluidized-bed drying or spray-granulation,
wherein steps C., D., E. and F. can be carried out at any time during the process.

Embodiment 55: Process for making a suspension concentrate (SC) formulation comprising:
d. providing biomass comprising spores of fusaricidin producing bacteria,
e. dispersing said biomass by shear or mechanical energy input, e.g. in a rotor-stator colloid mill or a bead mill in a continuous phase comprising,
f. adding at least one category 1 additive,
g. optionally adding at least one category 2 additive,
h. optionally adding at least one category 3 additive,
i. optionally adding auxiliaries,
j. wherein steps c), d), e) and f) can be carried out at any time during the process.

### EXAMPLES

Materials:

| | | | |
|---|---|---|---|
| Product Name | Function | Category | Chemical name |

| | | |
|---|---|---|
| Wettol ® D3 | Wetting Agent, | naphthalenesulfonic acid-formaldehyde- |
| | Dispersant | polycondensate, sodium salt |
| Polyfon ® H | Dispersant | Kraft-Lignin-Sulfonate, aliphatic |
| | | sulfonate, low Sulfonate, high Mw |
| Polyfon ® F | Dispersant | Kraft-Lignin-Sulfonate, aliphatic |
| | | sulfonate, high Sulfonate, high |
| | | Mw |
| Borresperse Na | Dispersant | Lignosulfonic acid, sodium salt |
| Atlox ™ 4913 | Dispersant | Methyl methacrylate graft copolymer, |
| | | anionic (solution) |
| Atlox ™ 4915 | Dispersant | Low charge PEG-quat-C15-triacid |
| Atlox ™ 4916 | Dispersant | Sorbitol ethoxylate ester of polymerized |
| | | fatty acid |
| Borresperse Ca | Dispersant | Lignosulfonic acid, calcium salt |
| Vanisperse ® CB | Dispersant | Lignosulfonic acid, sodium salt |
| Wettol ® D1 | Wetting agent, | Benzenesulfonic acid-formaldehyde- |
| | dispersant | phenol-urea-polycondensate, |
| | | sodium salt |
| Tensiofix ® BCZ | Wetting agent | Sodium lauryl sulfate |
| Agnique ® SLES | Wetting agent | Sodium lauryl ether sulfate (solution) |
| 370 | | |
| Agnique ® PG 8107 | Wetting agent, | Alkyl polyglucoside (solution) |
| | dispersant | |
| Tween ® 80 | Wetting agent, | Poly(oxyethylene) (20) sorbitan |
| | dispersant | monooleate |
| Sokalan ® HP50 | Dispersant | Polyvinylpyrrolidone, Mw 40 000 |
| Pluronic ® PE 3100 | Dispersant | Polyethylene glycol - polypropylene |
| | | glycol block polymer, approx. |
| | | 10% ethylene glycol |
| Pluronic ® PE 10500 | Dispersant | Polyethylene glycol - polypropylene |
| | | glycol block polymer, approx. |
| | | 50% ethylene glycol |
| Pluronic ® PE 6200 | Dispersant | Polyethylene glycol - polypropylene |
| | | glycol block polymer, approx. |
| | | 20% ethylene glycol |
| Pluronic ® PE 6400 | Dispersant | Polyethylene glycol - polypropylene |
| | | glycol block polymer, approx. |
| | | 40% ethylene glycol |
| Luvitec ® VA 64 | Dispersant | Statistical copolymer of vinyl pyrrolidone |
| | | and vinyl acetate |
| Break-Thru ® SP133 | Wetting agent, | Mixture of polyglycerolesters, fatty |
| | dispersant | acid esters |
| Span ™ 80 | Dispersant | Sorbitan monooleate |
| Tween ® 20 | Dispersant | Poly oxyethylene (20) sorbitan |
| | | monolaurate |
| Break-Thru ® SF | Dispersant | Aqueous solution of sophorolipid |
| 420 | | |
| Brij ®-23 | Dispersant | Polyoxyethylene (23) lauryl ether |
| Atlas ™ G-1086 | Dispersant | Polyoxyethylene (40) sorbitol hexaoleate |
| Atlas ™ G-1096 | Dispersant | Sorbitol ethoxylate esters |
| Dispersogen PSL | Dispersant | Polyacrylates graft copolymer, nonionic |
| 100 | | |
| Sokalan ® HP20 | Dispersant | Polyethyleneimine, ethoxylated, |
| | | cationic |
| Break-Thru ® EM | Wetting agent | Polyglycerol oleic acid esters, |
| O5 | | HLB 5.5 |
| Lutensol ® XL70 | Wetting agent | Alkyl polyethylene glycol ether |
| | | from C10-Guerbet alcohol, HLB |
| | | 13 |
| Lutensol ® XL140 | Dispersant | Alkyl polyethylene glycol ether |
| | | from C10-Guerbet alcohol, HLB |
| | | 16 |
| Lutensol ® AT 80 | Dispersant | Alkyl polyethylene glycol ether |
| | | from C16 C18 fatty alcohol, HLB |
| | | 18.5 |
| Lutensol ® AT 11 | Dispersant | Alkyl polyethylene glycol ether |
| | | from C16 C18 fatty alcohol, HLB |
| | | 13 |
| Sipernat ® 310 | Insoluble filler | Finely ground precipitated silica |
| Pluriol ® E200 | Diluent | Polyethylene glycol, molecular |
| | | weight 200 Dalton |
| Sunflower oil | Diluent | Sunflower oil, refined |
| Ammonium sulfate | Soluble filler | Ammonium sulfate |
| Celite ® 266 | Insoluble filler | Diatomaceous earth |
| Sipernat ® 310 | Insoluble filler | Precipiated silica |
| Magensium sulfate - | Soluble filler | Magensium sulfate - heptahydrate |
| heptahydrate | | |
| Potassium phosphate, | Soluble filler | Potassium phosphate, dibasic |
| dibasic | | |
| Potassium phosphate, | Soluble filler | Potassium phosphate, monobasic |
| monobasic | | |
| Wacker ® Silicone | Antifoam | Polydimethylsiloxane emulsion |
| SRE-PFL | | |
| Break-Thru ® S301 | Wetting agent | Siloxane surfactant |
| Aerosil ® 200 | Anti-settling agent | Fumed silica, hydrophilic |
| Wettol ® D3 | Wetting Agent, | naphthalenesulfonic acid-formaldehyde- |
| | Dispersant | polycondensate, sodium salt |
| Polyfon ® H | Dispersant | Kraft-Lignin-Sulfonate, aliphatic |
| | | sulfonate, low Sulfonate, high Mw |

### Example 1: Greenhouse Tests of Paenibacillus sp. Strain Lu17007 Powder Tank-Mixed with Different Additive Combinations

Paenibacillus Technical Active Ingredient

Paenibacillus sp. strain Lu17007, isolated 1996 in Stuttgart (Germany), deposited with DSMZ under Accession No. DSM 26970 and disclosed in WO2016/020371, was grown in a complex medium to an OD of 25 to 50 and a spore content of 5×10⁷ CFU/ml to 5×10⁸ CFU/ml. The whole fermentation broth was washed by diafiltration. The resulting pellet was mixed with finely ground precipitated silica. This suspension was spray dried under nitrogen atmosphere at 150° C. to 160° C. inlet temperature and 80° C. to 95° C. outlet temperature.

The resulting fine powder of Paenibacillus sp. strain Lu17007 containing 9% precipitated silica (w/w with respect to the total dry mass of the resulting dry product) was used as technical active ingredient (TGAI) in the following experiments.

Wettable Powder Premix and Additive Tank-Mix Solutions

Paenibacillus sp. strain Lu17007 TGAI was dry mixed with fillers to create wettable powder premixes. Wettable powder premix P.11 consisted of 50% (w/w) Paenibacillus sp. strain Lu17007 TGAI, 45% Ammonium sulfate and additional 5% of finely ground precipitated silica. WP premix P.12 consisted of 50% Paenibacillus sp. strain Lu17007 TGAI, 25% Ammonium sulfate and 25% diatomaceous earth as fillers.

Dilution media for spray suspensions were prepared by adding wetting agents and dispersants to tap water and homogenizing. Dilution medium A.1 contained 0.05% (w/w) sodium salt of naphthalene sulfonic acid-formaldehyde polycondensate and 0.01% sodium lauryl sulfate. Dilution medium N.1 contained 0.05% polyoxyethylene (20) sorbitan monooleate and 0.05% sorbitan monooleate. The wettable powder premixes P.11 and P.12 were dosed into the dilution media A.1 and N.1 respectively as shown in table 1, and shortly stirred to create homogeneous spray slurries.

Greenhouse Pot Trials

The fungicidal activity of the resulting spray suspensions containing Paenibacillus sp. strain Lu17007 wettable powder and additives as listed in table 1, was compared in greenhouse pot trials at the BASF trial station in Limburgerhof/Germany. Tests were done against Phytophthora infestans and Alternaria Solani, both in tomatoes cv. Goldene Königin, and against Botrytis cinerea in pepper cv. Neusiedler Ideal. The plants were used in BBCH stage 12 (=2nd leaf unfolded). A spraying cabinet was used for the application on plants. All trials had 3 replicates and the plots were randomized. One day after the application of the products, the trials were inoculated by using a spore suspension of the pathogen (2E+5 spores/mL). Incubation took place in the greenhouse for 3 to 5 d at 21° C. and 90% rH before the evaluation of percent leaf damage was carried out.

TABLE 1
Antifungal activity of Paenibacillus sp. strain Lu17007 WP formulation
combined with different additives as tank-mix (dose rate in % w/w).

| | | Dose rate WP | | Efficacy | Efficacy | Efficacy |
|---|---|---|---|---|---|---|
| Spray | | premix (% | | Efficacy | Efficacy | Efficacy |
| slurry/ | WP | w/w of total | Dilution | ALTESO | BOTRCI | PHYTIN |
| treatment | premix | spray slurry) | medium | % | % | % |
| 1 | None | 0 | Tab water | 0 | 0 | 0 |
| 2 | Paenibacillus | 0.044 | Tab water | 90 | 72 | 73 |
| | sp. strain | | | | | |
| | Lu17007 | | | | | |
| | TGAI | | | | | |
| 3 | P.11 | 0.044 | A.1 | 29 | 5 | 13 |
| 4 | P.11 | 0.067 | A.1 | 50 | 29 | 23 |
| 5 | P.11 | 0.111 | A.1 | 86 | 35 | 76 |
| 6 | P.11 | 0.044 | N.1 | 87 | 84 | 72 |
| 7 | P.11 | 0.067 | N.1 | 95 | 92 | 80 |
| 8 | P.11 | 0.111 | N.1 | 97 | 99 | 80 |
| 9 | P.12 | 0.044 | A.1 | 21 | 0 | 10 |
| 10 | P.12 | 0.067 | A.1 | 36 | 0 | 47 |
| 11 | P.12 | 0.111 | A.1 | 93 | 0 | 88 |
| 12 | P.12 | 0.044 | N.1 | 96 | 71 | 65 |
| 13 | P.12 | 0.067 | N.1 | 99 | 83 | 72 |

The treatments containing dilution medium N.1 with nonionic wetting agent and dispersant performed significantly better against Alternaria solani on tomato, Botrytis cinerea on pepper and Phytophtera infenstans on tomato at much lower active ingredient rate than treatments containing dilution medium A.1 with anionic wetting agent and dispersant.

### Example 2: Greenhouse Pot Trials with Paenibacillus sp. Strain A Formulations from Concentrated Broth

Paenibacillus sp. strain A, a mutant of Paenibacillus sp. strain Lu17007, was grown in a complex medium to an OD of 25 to 50 and a spore content of 5×10⁷ CFU/ml to 5×10⁸ CFU/ml. The whole fermentation broth was run over a disc separator to create a pellet with 8.9% dry mass. The pellet fraction was mixed with formulation additives as listed in table 2.

TABLE 2
Liquid Paenibacillus sp. strain A formulations.
Amount of ingredients is given in g/kg.

| Ingredient (g/kg) | P.2 | N.21 | N.22 | A.21 | A.22 |
|---|---|---|---|---|---|
| Paenibacillus sp. | 562 | 562 | 562 | 562 | 562 |
| strain A pellet | | | | | |
| Sipernat ® 310 | | 112 | 112 | | |
| Celite ® 266 | | 304 | | 225 | 304 |
| Ammonium sulfate | | 0 | 304 | 180 | |
| Tensiofix ® BCZ | | | | 11 | |
| Polyfon ® F | | | | | 56 |
| Polyfon ® H | | | | | 112 |
| Borresperse Na | | | | 90 | |
| Pluronic ® PE 3100 | | 56 | 56 | | |
| Sokalan ® HP50 | | 56 | 56 | | |
| Tween ® 80 | | | | | 28 |
| Sunflower oil | | | | | 28 |
| Wacker ® Silicone | | | | 22 | |
| SRE-PFL | | | | | |
| Water | 562 | 34 | 34 | 34 | 34 |

The liquid Paenibacillus sp. strain A formulations of table 2 were diluted with tap water to the desired concentration as listed in

Table 3 for application in greenhouse pot trials against Alternaria solani on tomato, Botrytis cinerea on pepper and Phytophtera infenstans on tomato, as described in detail in example 1.

TABLE 3
Antifungal activity of liquid Paenibacillus sp. strain A formulations.

| | | Dose rate | | | | |
|---|---|---|---|---|---|---|
| | | liquid premix | | | | |
| Spray | | (% w/w of | | Efficacy | Efficacy | Efficacy |
| slurry/ | | total spray | Dilution | ALTESO | BOTRCI | PHYTIN |
| treatment | Premix | slurry) | medium | % | % | % |
| 1 | Control | none | Tab water | 0 | 0 | 0 |
| 2 | P.2 | 0.439 | Tab water | 46 | 3 | 3 |
| 3 | P.2 | 0.878 | Tab water | 56 | 7 | 14 |
| 4 | P.2 | 1.755 | Tab water | 84 | 13 | 57 |
| 5 | N.21 | 0.439 | Tab water | 80 | 10 | 21 |
| 6 | N.21 | 0.878 | Tab water | 96 | 73 | 46 |
| 7 | N.21 | 1.755 | Tab water | 94 | 100 | 77 |
| 8 | N.22 | 0.439 | Tab water | 80 | 15 | 46 |
| 9 | N.22 | 0.878 | Tab water | 92 | 50 | 53 |
| 10 | N.22 | 1.755 | Tab water | 99 | 91 | 77 |
| 11 | A.21 | 0.439 | Tab water | 75 | 7 | 14 |
| 12 | A.21 | 0.878 | Tab water | 85 | 13 | 32 |
| 13 | A.21 | 1.755 | Tab water | 94 | 15 | 62 |
| 14 | A.22 | 0.439 | Tab water | 68 | 20 | 3 |
| 15 | A.22 | 0.878 | Tab water | 73 | 13 | 10 |
| 16 | A.22 | 1.755 | Tab water | 87 | 60 | 17 |

The treatments containing formulations N.21 and N.22 with nonionic wetting agents and dispersants performed significantly better at the two highest dose rates against Botrytis cinerea and Phytophtera infenstans than the diluted Paenibacillus sp. strain A pellet alone or the treatments containing formulations A.21 and A.22 with anionic wetting agents and dispersants. For Alternaria solani, the effect was not so pronounced, since the infection level in this trial was extremely low (67.5%).

### Example 3: Greenhouse Activity of Paenibacillus sp. Strain B Powder in Tank-Mix with Different Surface-Active Additives

Paenibacillus sp. strain B, a mutant of Paenibacillus sp. strain Lu17007, was grown was grown in a complex medium to an OD of 25 to 50 and a spore content of 5×10⁷ CFU/ml to 5×10⁸ CFU/ml. The whole fermentation broth was run over a disc separator to create a pellet with 2.3% dry mass. The pellet fraction was mixed with finely ground precipitated silica and the suspension was spray dried under nitrogen atmosphere with an inlet temperature of 170° C. to 180° C. and an outlet temperature of 89° C. to 94° C. The resulting fine powder of Paenibacillus sp. strain B containing 10% precipitated silica (w/w with respect to the total dry mass of the resulting dry product) was used as technical active ingredient (TGAI) in the following examples 4 to 6. In Table 4 this TGAI is referenced to as P.5.

Paenibacillus sp. strain B TGAI, referenced as P.5, was tank-mixed with various formulation additives diluted in tab water as listed in Table 4. The resulting spray slurry was tested against Alternaria solani on tomato, Botrytis cinerea on pepper and Phytophtera infenstans on tomato in greenhouse pot trials as described in detail in example 1.

TABLE 4
Antifungal activity of Paenibacillus sp. strain B TGAI combined
with tank-mix additives at different concentrations (% w/w).

| | Dose | | | | | |
|---|---|---|---|---|---|---|
| | rate of | | Additive | Efficacy | Efficacy | Efficacy |
| Spray | TAGI | Additive in | concen- | Efficacy | Efficacy | Efficacy |
| slurry/ | P.5 % | dilution | tration | ALTESO | BOTRCI | PHYTIN |
| treatment | w/w | medium | % w/w | % | % | % |
| 1 | control | None | | 0 | 0 | 0 |
| 2 | 0.056 | None | | 89 | 89 | 92 |
| 3 | 0.056 | Tensiofix ® BCZ | 0.01 | 94 | 80 | 77 |
| 4 | 0.056 | Wettol ® D1 | 0.01 | 11 | 2 | 2 |
| 5 | 0.056 | Agnique ® SLES 370 | 0.01 | 74 | 83 | 75 |
| 6 | 0.056 | Polyfon ® H | 0.1 | 53 | 42 | 0 |
| 7 | 0.056 | Wettol ® D3 | 0.1 | 15 | 0 | 2 |
| 8 | 0.056 | Borresperse Na | 0.1 | 53 | 66 | 47 |
| 9 | 0.056 | Sokalan ® HP 50 | 0.1 | 83 | 97 | 79 |
| 10 | 0.056 | Pluronic ® PE3100 | 0.1 | 83 | 95 | 85 |
| 11 | 0.056 | Break-Thru ® SP133 | 0.1 | 90 | 95 | 77 |
| 12 | 0.111 | Agnique ® SLES 370 | 0.03 | 90 | 95 | 84 |
| 13 | 0.111 | Borresperse Na | 0.2 | 86 | 70 | 68 |
| 14 | 0.111 | Sokalan ® HP 50 | 0.2 | 98 | 95 | 99 |
| 15 | 0.111 | Pluronic ® PE3100 | 0.2 | 99 | 89 | 98 |
| 16 | 0.111 | Break-Thru ® SP133 | 0.2 | 99 | 99 | 100 |

Treatments with polyvinyl pyrrolidone, polyethylene—polypropylene glycol block copolymer, polyglycerol ester or with lauryl ether sulfate in the spray slurry performed much better than treatments containing other anionic wetting or dispersing agents (lauryl sulfate, naphthalene sulfonic acid co-condensate, lignosulfonates) usually used in dry and liquid crop protection formulations and well known to the skilled person.

### Example 4: Greenhouse Screening for Compatible Additives with Paenibacillus sp. Strain B Powder in Tank-Mix

Paenibacillus sp. strain B TGAI from example 3, referenced as P.5, was tank-mixed at a concentration of 0.083% with formulation additives diluted in tab water at two different concentrations as listed in Table 5. The resulting spray slurry was tested against Phytophtora infestans on tomatoes in greenhouse pot trials as described in detail in example 1. Table 5 shows the test results in relation to TGAI P.5 diluted in water.

TABLE 5
Activity of Paenibacillus sp. strain B TGAI combined with
various tank-mix additives against Phytophtora infestans.
Additives providing improved efficacy are highlighted in
bold. TGAI concentration is 0.083% w/w in all cases.

| | | Additive | Efficacy |
|---|---|---|---|
| Test | Additive in | concentration % | PHYTIN |
| No. | dilution medium | (w/w) | % |
| 1 | none | 0.000 | 65 |
| 1 | Atlox ™ 4913 | 0.020 | 44 |
| 1 | Atlox ™ 4913 | 0.150 | 77 |
| 1 | Atlox ™ 4915 | 0.020 | 20 |
| 1 | Atlox ™ 4915 | 0.150 | 48 |
| 1 | Atlox ™ 4916 | 0.020 | 41 |
| 1 | Atlox ™ 4916 | 0.150 | 87 |
| 1 | Luvitec ® VA64 | 0.020 | 84 |
| 1 | Luvitec ® VA64 | 0.150 | 88 |
| 1 | Pluronic ® PE10500 | 0.020 | 77 |
| 1 | Pluronic ® PE10500 | 0.150 | 94 |
| 1 | Pluronic ® PE6400 | 0.020 | 65 |
| 1 | Pluronic ® PE6400 | 0.150 | 97 |
| 1 | Borresperse Ca | 0.020 | 23 |
| 1 | Borresperse Ca | 0.150 | 41 |
| 2 | none | 0.000 | 50 |
| 2 | Lutensol ® AT 11 | 0.020 | 31 |
| 2 | Lutensol ® AT 11 | 0.150 | 50 |
| 2 | Lutensol ® AT 80 | 0.020 | 50 |
| 2 | Lutensol ® AT 80 | 0.150 | 81 |
| 2 | Lutensol ® XL 70 | 0.020 | 68 |
| 2 | Lutensol ® XL 70 | 0.150 | 68 |
| 2 | Lutensol ® XL 140 | 0.020 | 47 |
| 2 | Lutensol ® XL 140 | 0.150 | 65 |
| 2 | Break-Thru ® EM O5 | 0.020 | 59 |
| 2 | Break-Thru ® EM O5 | 0.150 | 68 |
| 2 | Borresperse Na | 0.020 | 33 |
| 2 | Borresperse Na | 0.150 | 29 |
| 3 | none | 0.000 | 26 |
| 3 | Tween ® 20 | 0.020 | 22 |
| 3 | Tween ® 20 | 0.150 | 44 |
| 3 | Break-Thru ® SF 420 | 0.020 | 44 |
| 3 | Break-Thru ® SF 420 | 0.150 | 24 |
| 3 | Atlas ™ G-1086 | 0.020 | 59 |
| 3 | Atlas ™ G-1086 | 0.150 | 28 |
| 3 | Atlas ™ G-1096 | 0.020 | 59 |
| 3 | Atlas ™ G-1096 | 0.150 | 48 |
| 3 | Dispersogen PSL 100 | 0.020 | 24 |
| 3 | Dispersogen PSL 100 | 0.150 | 17 |
| 3 | Sokalan ® HP 20 | 0.020 | 31 |
| 3 | Sokalan ® HP 20 | 0.150 | 56 |
| 4 | none | 0 | 45 |
| 4 | Tensiofix ® BCZ | 0.02 | 38 |
| 4 | Tensiofix ® BCZ | 0.15 | 13 |
| 4 | Polyfon ® F | 0.02 | 31 |
| 4 | Polyfon ® F | 0.15 | 16 |
| 4 | Wettol ® D1 | 0.02 | 42 |
| 4 | Wettol ® D1 | 0.15 | 5 |
| 4 | Brij ®-23 | 0.02 | 36 |
| 4 | Brij ®-23 | 0.15 | 56 |
| 4 | Span ™ 80 | 0.02 | 56 |
| 4 | Span ™ 80 | 0.15 | 5 |
| 4 | Angique SLES 370 | 0.02 | 56 |
| 4 | Angique SLES 370 | 0.15 | 73 |
| 4 | Borresperse Ca | 0.02 | 64 |
| 4 | Borresperse Ca | 0.15 | 24 |

The greenhouse results of Table 5 show that mainly nonionic additives positively influence the biological efficacy in a consistent way. Especially the additives from the chemical groups of polyethylene—polypropylene glycol block polymers (irrespective of hydrophobic-hydrophilic ratio and molecular weight), the group of alcohol ethoxylates and nonionic polymers or copolymers of vinylpyrrolidone provide a positive effect on biological efficacy. Also, Glyceryl oleate, Polyoxyethylene sorbitol hexaoleates and Polyoxyethylene (20) sorbitol monooleate, as well as ethoxylated polyethylene imine improved the efficacy on Phytophtora infestans in greenhouse pot trials when added to the spray slurry.

### Example 5: Greenhouse Test for Compatible Additives with Paenibacillus sp. Strain C

Paenibacillus sp. strain C, a mutant of Paenibacillus sp. strain Lu17007, was grown in a complex medium to an OD of 25 to 50 and a spore content of 5×10⁷ CFU/ml to 5×10⁸ CFU/ml. The whole fermentation broth was processed by microfiltration and concentrated to create a pellet with 5.5% dry mass. The pellet fraction was mixed with finely ground precipitated silica and the suspension was spray dried under nitrogen atmosphere with an inlet temperature of 170° C. to 180° C. and an outlet temperature of 85° C. to 95° C. The resulting fine powder of Paenibacillus sp. strain C containing 10% precipitated silica (w/w with respect to the total dry mass of the resulting dry product) was used as technical active ingredient (TGAI) in the following example.

Paenibacillus sp. strain C TGAI was tank-mixed at a concentration of 0.029% with formulation additives diluted in tab water at two different concentrations as listed in table 6. These spray slurries were tested as treatment against Phytophtora infestans on tomatoes in greenhouse pot trials.

Also, for strain C, polyethylene—polypropylene glycol block polymers Pluronic® PE10500 and PE6200 significantly improved biological efficacy against Phytophtora infestans. For alkylpolyglucoside Agnique® PG 8107 and sodium lauryl ether sulfate Agnique® SLES the impact on efficacy depends on the concentration. While for the neutral alkylpolyglucoside Agnique® PG8107 the higher concentration was beneficial, the lower concentration was advantageous for the anionic surfactant sodium lauryl ether sulfate.

TABLE 6
Activity of Paenibacillus sp. strain C TGAI combined with
various tank-mix additives against Phytophtora infestans.
Additives providing improved efficacy are highlighted in
bold. TGAI concentration is 0.029% w/w in all cases.

| | | Additive | Efficacy |
|---|---|---|---|
| Treatment | Additive in | concentration % | PHYTIN |
| No. | dilution medium | (w/w) | % |
| 1 | none | 0.000 | 65 |
| 2 | Pluronic ® PE10500 | 0.020 | 92 |
| 3 | Pluronic ® PE10500 | 0.150 | 99 |
| 4 | Pluronic ® PE6200 | 0.020 | 92 |
| 5 | Pluronic ® PE6200 | 0.150 | 98 |
| 6 | Vanisperse ® CB | 0.020 | 32 |
| 7 | Vanisperse ® CB | 0.150 | 9 |
| 8 | Agnique ® PG 8107 | 0.020 | 53 |
| 9 | Agnique ® PG 8107 | 0.150 | 92 |
| 10 | Agnique ® SLES-370 | 0.020 | 87 |
| 11 | Agnique ® SLES-370 | 0.150 | 68 |

### Example 6: Wettable Powder Formulations of Paenibacillus sp. Strain C and their Greenhouse Activity

Paenibacillus sp. strain C was grown was grown in a complex medium to an OD of 25 to 50 and a spore content of 5×10⁷ CFU/ml to 5×10⁸ CFU/ml. The whole fermentation broth was processed by microfiltration and concentrated to create a pellet with 5.5% dry mass. The pellet fraction was mixed with finely ground precipitated silica and the suspension was spray dried under nitrogen atmosphere with an inlet temperature of 110° C. to 140° C. and an outlet temperature of 60° C. to 70° C. The resulting fine powder of Paenibacillus sp. strain C containing 10% precipitated silica (w/w with respect to the total dry mass of the resulting dry product) was used as technical active ingredient (TGAI) in the following example.

Further wettable powders were produced by adding formulation additives to the pellet fraction according to the recipes in table 7, followed by spray drying with the parameters mentioned above.

TABLE 7
Wettable powder formulations of Paenibacillus sp. strain C.

| Ingredient | | | | | | | | | | |
|---|---|---|---|---|---|---|---|---|---|---|
| (% w/w) | Function | TGAI | WP1 | WP2 | WP3 | WP4 | WP5 | WP6 | WP7 | WP8 |
| Bio dry mass | | 90 | 30 | 30 | 30 | 30 | 31 | 30 | 30 | 30 |
| Sipernat ® | Insoluble | 10 | 30 | | 10 | 10 | 10 | 10 | 28 | 10 |
| 310 | filler | | | | | | | | | |
| Chalk | Insoluble | | | 30 | | | | | | |
| (calcium | filler | | | | | | | | | |
| carbonate) | | | | | | | | | | |
| Ammonium | Soluble | | 20 | 20 | 20 | | | | 20 | |
| sulfate | filler | | | | | | | | | |
| Magnesium | Soluble | | | | | 20 | | | | |
| sulfate | filler | | | | | | | | | |
| Potassium | Soluble | | | | | | 12 | | | |
| phosphate | filler | | | | | | | | | |
| monobasic | | | | | | | | | | |
| Potassium | Soluble | | | | | | 6 | | | |
| phosphate | filler | | | | | | | | | |
| dibasic | | | | | | | | | | |
| Pluronic ® | Category 1 | | 10 | 10 | 20 | 20 | 20 | 10 | 10 | 10 |
| PE 10500 | additive | | | | | | | | | |
| Pluronic ® | Category 1 | | 10 | 10 | 19 | 19 | 20 | 10 | 10 | 10 |
| PE 6200 | additive | | | | | | | | | |
| Agnique ® | Category 2 | | | | 1 | 1 | 1 | 1 | | 1 |
| SLES 370 | additive | | | | | | | | | |
| Borresperse | Category 3 | | | | | | | | | 39 |
| Na | additive | | | | | | | | | |
| Vanisperse ® | Category 3 | | | | | | | 39 | 2 | |
| CB | additive | | | | | | | | | |

Spray slurries produced from the wettable powder formulations as listed in table 7, containing 0.028% w/w to 0.087% w/w formulation were tested as treatment against Phytophtora infestans on tomatoes and Botrytis cinerea on pepper in greenhouse pot trials, as describe in detail in example 1. From the results listed in table 8, it is clearly visible that WP 1 to WP 5, containing additives of category 1, show superior biological efficacy compared to the TGAI at the same biomass content. On the other hand, WP 6 containing substantial amount of anionic sulfonated dispersing agent almost lost its efficacy, while WP 7 with a significantly lower amount of anionic sulfated dispersing agent is still inferior to WP 1 to WP 5.

TABLE 8
Activity of Paenibacillus sp. strain C wettable powder formulations
against Phytophtora infestans and Botrytis cinerea.

| | | | | Efficacy | Efficacy |
|---|---|---|---|---|---|
| Treatment | Formulation | % w/w | % w/w | PHYTIN | BOTRCI |
| No | No | formulation | biomass | % | % |
| 1 | TGAI | 0.028 | 0.025 | 43 | 53 |
| 2 | WP1 | 0.087 | 0.026 | 84 | 92 |
| 4 | WP2 | 0.087 | 0.026 | 80 | 68 |
| 6 | WP3 | 0.087 | 0.026 | 87 | 76 |
| 8 | WP4 | 0.087 | 0.026 | 88 | 94 |
| 10 | WP5 | 0.087 | 0.026 | 89 | 92 |
| 12 | WP6 | 0.087 | 0.026 | 14 | 23 |
| 14 | WP7 | 0.087 | 0.026 | 61 | 90 |
| 3 | WP1 | 0.174 | 0.052 | 88 | 100 |
| 5 | WP2 | 0.174 | 0.052 | 96 | 100 |
| 7 | WP3 | 0.174 | 0.052 | 89 | 97 |
| 9 | WP4 | 0.174 | 0.052 | 98 | 100 |
| 11 | WP5 | 0.174 | 0.052 | 94 | 98 |
| 13 | WP6 | 0.174 | 0.052 | 32 | 31 |
| 15 | WP7 | 0.174 | 0.052 | 84 | 99 |

### Example 7: Wettable Powder Formulations of Paenibacillus sp. Strain C and their Greenhouse Activity

Further wettable powders listed in table 9 were produced by adding formulation additives to Paenibacillus polymyxa C pellet fraction followed by spray-drying according to the procedure described in example 6.

TABLE 9
Wettable powder formulations of Paenibacillus sp. strain C.

| Ingredient (% w/w) | Function | WP9 | WP10 | WP11 | TGAI |
|---|---|---|---|---|---|
| Bio dry mass | | 30 | 30 | 30 | 90 |
| Sipernat ® 310 | Insoluble filler | 17 | 30 | 23 | 10 |
| Ammonium sulfate | Soluble filler | 20 | 20 | 20 | |
| Break Thru S 301 | Wetting agent | 2 | | 2 | |
| Break Thru SP 133 | Category 1 | | | 5 | |
| | additive | | | | |
| Pluronic ® | Category 1 | 13 | 10 | 15 | |
| PE10500 | additive | | | | |
| Pluronic ® PE 6200 | Category 1 | 13 | 10 | | |
| | additive | | | | |
| Atlox ™ 4913 | Category 2 | 5 | | 5 | |
| | additive | | | | |

Spray slurries produced from dilutions of the wettable powder formulations of table 8 and table 9 in tap water, containing 0.017% w/w to 0.051% w/w formulation, were tested as treatment against Phytophtora infestans on tomatoes and Botrytis cinerea on pepper in greenhouse pot trials as described in example 1. The results are listed in table 10. It can be clearly seen that WP9, WP10 and WP11 containing the favorable formulation additives of category 1 show a much better performance than the TGAI, while formulations WP6 and WP8 containing category 3 additives in a substantial amount have significantly inferior biological efficacy.

TABLE 10
Activity of Paenibacillus sp. strain C wettable powder formulations against
Alternaria solani, Phytophtora infestans and Botrytis cinerea.

| | | | | Efficacy | Efficacy | Efficacy |
|---|---|---|---|---|---|---|
| Treatment | Formulation | % w/w | % w/w | ALTESO | PHYTIN | BOTRCI |
| No | No. | Formulation | biomass | % | % | % |
| 1 | TGAI | 0.017 | 0.015 | 41 | 55 | 61 |
| 2 | TGAI | 0.034 | 0.031 | 92 | 87 | 88 |
| 3 | TGAI | 0.051 | 0.046 | 99 | 99 | 100 |
| 4 | WP10 | 0.051 | 0.015 | 73 | 85 | 92 |
| 5 | WP10 | 0.102 | 0.031 | 84 | 100 | 92 |
| 6 | WP6 | 0.037 | 0.011 | 0 | 0 | 3 |
| 7 | WP6 | 0.074 | 0.022 | 0 | 82 | 21 |
| 8 | WP8 | 0.037 | 0.011 | 0 | 10 | 21 |
| 9 | WP8 | 0.074 | 0.022 | 36 | 17 | 72 |
| 10 | WP9 | 0.051 | 0.015 | 63 | 87 | 80 |
| 11 | WP9 | 0.102 | 0.031 | 90 | 93 | 96 |
| 12 | WP9 | 0.153 | 0.046 | 95 | 100 | 98 |
| 13 | WP11 | 0.051 | 0.015 | 75 | 79 | 90 |
| 14 | WP11 | 0.102 | 0.031 | 92 | 99 | 98 |
| 15 | WP11 | 0.153 | 0.046 | 98 | 99 | 97 |

### Example 8 Suspension Concentrate Formulations of Paenibacillus sp. Strain C and their Greenhouse Activity

TGAI of Paenibacillus sp. strain C containing 10% precipitated silica was prepared as described in example 5. To prepare a suspension concentrate formulation, water and PEG 200 were mixed on a 1:1 w/w basis. After adding the other formulation ingredients listed in table 11, the TGAI was stirred in with low shear until a homogeneous mixture was achieved.

TABLE 11
Suspension concentrate formulations of Paenibacillus sp. strain C.

| Ingredient | | | | | | |
|---|---|---|---|---|---|---|
| (g/L) | Function | SC1 | SC2 | SC3 | SC4 | SC5 |
| TGAI | | 200 | 200 | 200 | 200 | 200 |
| Water | Diluent | 450 | 415 | 390 | 390 | 390 |
| PEG 200 | Diluent | 450 | 415 | 390 | 390 | 390 |
| Pluronic ® | Category 1 | | | 100 | 70 | 50 |
| PE 10500 | additive | | | | | |
| Pluronic ® | Category 1 | | | | | 50 |
| PE 6400 | additive | | | | | |
| Pluronic ® | Category 1 | | 50 | | | |
| PE 6200 | additive | | | | | |
| Break-Thru ® | Category 1 | | | | 30 | |
| SP 133 | additive | | | | | |
| Atlox ™ 4913 | Category 2 | | 20 | 20 | 20 | 20 |
| | additive | | | | | |

Spray slurries were produced from the suspension concentrate formulations of table 11 by dilution in tap water, containing 0,107% w/w and 0,160% w/w of formulation. The fungicidal activity of the resulting spray suspensions was compared in greenhouse pot trials against Phytophthora infestans and Alternaria Solani, both in tomatoes, and against Botrytis cinerea in pepper, as previously described in detail in example 1. The results are listed in table 12. While the SC1 with no further formulation additives does not perform well in ALTESO and PHYTIN, SC2 to SC4 containing advantageous formulation additives of category 1 and 2, have significantly enhanced biological performance, also on BOTRCI.

TABLE 12
Activity of Paenibacillus sp. strain C suspension
concentrate formulations against Alternaria solani,
Phytophtora infestans and Botrytis cinerea.

| | | % w/w | Efficacy | Efficacy | Efficacy |
|---|---|---|---|---|---|
| Treatment | Formula- | formula- | ALTESO | BOTRCI | PHYTIN |
| No | tion No | tion | % | % | % |
| 1 | SC1 | 0.107 | 70 | 59 | 29 |
| 2 | SC1 | 0.160 | 38 | 90 | 5 |
| 3 | SC2 | 0.107 | 88 | 78 | 49 |
| 4 | SC2 | 0.160 | 88 | 99 | 76 |
| 5 | SC3 | 0.107 | 90 | 77 | 61 |
| 6 | SC3 | 0.160 | 91 | 98 | 85 |
| 7 | SC4 | 0.107 | 92 | 73 | 83 |
| 8 | SC4 | 0.160 | 96 | 90 | 80 |
| 9 | SC5 | 0.107 | 90 | 77 | 73 |
| 10 | SC5 | 0.160 | 89 | 85 | 81 |

### Example 9: Non-Aqueous Suspension Concentrate Formulations of Paenibacillus sp. Strain C and their Greenhouse Activity

TGAI of Paenibacillus sp. strain C was prepared as described in example 5. Non-aqueous suspension concentrate formulations were prepared by mixing PEG 200 with the formulation additives listed in table 13, and as a last step stirring in 200 g/L TGAI with low shear until homogeneous, followed by one passage through a rotor-stator colloid mill IKA magic lab.

TABLE 13
Non-aqueous suspension concentrate formulations
of Paenibacillus sp. strain C.

| Ingredient (g/L) | Function | SC6 | SC7 | SC8 | SC9 |
|---|---|---|---|---|---|
| TGAI | | 200 | 200 | 200 | 200 |
| PEG 200 | Diluent | 900 | 820 | 760 | 780 |
| Pluronic ® PE | Category 1 | | | 50 | |
| 6400 | additive | | | | |
| Agnique ® | Category 1 | | | | 50 |
| PG8107 | additive | | | | |
| Atlox ™ 4913 | Category 2 | | 70 | 70 | 70 |
| | additive | | | | |
| Aerosil ® 200 | Anti-settling | | 10 | 20 | |
| | agent | | | | |

Spray slurries were produced from the suspension concentrate formulations of Table 13 by dilution in tap water, containing 0,107% w/w to 0,215% w/w of formulation. The fungicidal activity of the resulting spray suspensions was compared in greenhouse pot trials against Phytophthora infestans and Alternaria solani, both in tomato, and against Botrytis cinerea in pepper, as previously described in detail in Example 1. The results listed in Table 14 show that the non-aqueous suspension concentrate SC6 without additives has a good performance only at the highest application rate. Formulations with additives of category 1 and category 2, as SC7 to SC9, show further enhanced biological performance when compared to SC6, especially at the lower application rates.

TABLE 14
Activity of Paenibacillus sp. strain C non-aqueous suspension
concentrate formulations against Alternaria solani, Phytophtora infestans
and Botrytis cinerea.

| | | % w/w | Efficacy | Efficacy | Efficacy |
|---|---|---|---|---|---|
| Treatment | Formula- | Formula- | ALTESO | PHYTIN | BOTRCI |
| No | tion No | tion | % | % | % |
| 1 | SC6 | 0.107 | 44 | 36 | 69 |
| 2 | SC6 | 0.161 | 60 | 61 | 67 |
| 3 | SC6 | 0.215 | 78 | 82 | 76 |
| 4 | SC7 | 0.107 | 70 | 50 | 58 |
| 5 | SC7 | 0.161 | 87 | 72 | 81 |
| 6 | SC7 | 0.215 | 93 | 77 | 90 |
| 7 | SC8 | 0.107 | 58 | 56 | 71 |
| 8 | SC8 | 0.161 | 82 | 73 | 75 |
| 9 | SC8 | 0.215 | 90 | 76 | 91 |
| 10 | SC9 | 0.107 | 53 | 38 | 77 |
| 11 | SC9 | 0.161 | 82 | 56 | 85 |
| 12 | SC9 | 0.215 | 84 | 83 | 95 |

### Example 10: Non-Aqueous Suspension Concentrate Formulations with Various Liquid Media

TGAI of Paenibacillus sp. strain C was prepared as described in example 5. Non-aqueous suspension concentrate formulations were prepared by mixing PEG 200 (SC10), triethyl citrate (SC11) or hexylene glycol (SC12), with the formulation additives listed in table 15, and as a last step stirring in 170 g/kg TGAI with low shear until homogeneous, followed by one passage through a rotor-stator colloid mill IKA magic lab.

Formulation samples were put into incubation chambers at 40° C. and reevaluated after 8 and 16 weeks. The results, also shown in table 15, prove that all three formulations display sufficient stability at accelerated storage conditions with high temperatures.

TABLE 15
Non-aqueous suspension concentrate formulations of Paenibacillus sp. strain C.

| Ingredient (g/kg) | Function | SC10 | SC11 | SC12 |
|---|---|---|---|---|
| TGAI | | 170 | 170 | 170 |
| PEG 200 | Diluent | 710 | | |
| Triethyl citrate | Diluent | | 710 | |
| Hexylene glycol | Diluent | | | 710 |
| Pluronic ® PE 6400 | Category 1 additive | 20 | 20 | 20 |
| Atlox ™ 4913 | Category 2 additive | 85 | 85 | 85 |
| Aerosil ® 200 | Anti-settling agent | 10 | 10 | 10 |
| Struktol ® J673 A | Antifoam | 5 | 5 | 5 |

Initial physical parameters

| | | | | |
|---|---|---|---|---|
| Viable spores | cfu/ml | 2.8E+09 | 2.9E+09 | 3.6E+09 |
| Particle size d50/d90 | μm | 9.5/21.4 | 9.4/22.1 | 9.7/22.1 |
| Viscosity, 100/s | mPas | 224 | 178 | 160 |
| pH, 1% dilution | | 6.2 | 6.2 | 6.3 |

After 8 weeks 40° C.

| | | | | |
|---|---|---|---|---|
| Viable spores | cfu/ml | 3.4E+09 | 4.0E+09 | 4.6E+09 |
| Particle size d50/d90 | μm | 10.4/20.3 | 13.6/74.1 | 11.4/22.8 |
| Viscosity, 100/s | mPas | 256 | 195 | 238 |
| pH, 1% dilution | | 5.8 | 4.7 | 5.5 |

After 16 weeks 40° C.

| | | | | |
|---|---|---|---|---|
| Viable spores | cfu/ml | 3.0E+09 | 2.3E+09 | 1.4E+09 |
| Particle size d50/d90 | μm | 16.1/36.7 | 14.3/65.4 | 12.0/27.0 |
| Viscosity, 100/s | mPas | 333 | 198 | 306 |
| pH, 1% dilution | | 6.4 | 4.3 | 6 |
| Serum/sediment | | traces/none | traces/none | traces/none |
| Redispersibility | | good | good | good |

After 16 weeks of storage at 40° C., spray slurries were produced from the suspension concentrate formulations of Table 16 by dilution in tap water, containing 0.095% w/w to 0,196% w/w of formulation. The fungicidal activity of the resulting spray suspensions was compared in greenhouse pot trials against Phytophthora infestans and Alternaria solani; both in tomato, as previously described in detail in Example 1. The results listed in Table 16 show that the non-aqueous suspension concentrates with different dilution media SC10, SC11 and SC12 all display show good biological performance starting at the intermediate dose rate. SC10 and SC11 have a weaker performance than SC12 at lower dose rate.

TABLE 16
Activity of Paenibacillus sp. strain C non-aqueous
suspension concentrate formulations against Alternaria solani
and Phytophtora infestans

| | | | Efficacy | Efficacy |
|---|---|---|---|---|
| Treatment | Formulation | % w/w | ALTESO | PHYTIN |
| No | No | Formulation | % | % |
| 1 | SC10 | 0.095 | 43 | 51 |
| 2 | SC10 | 0.143 | 80 | 92 |
| 3 | SC10 | 0.190 | 89 | 82 |
| 4 | SC11 | 0.096 | 56 | 65 |
| 5 | SC11 | 0.144 | 83 | 86 |
| 6 | SC11 | 0.192 | 92 | 87 |
| 7 | SC12 | 0.098 | 78 | 76 |
| 8 | SC12 | 0.147 | 87 | 94 |
| 9 | SC12 | 0.196 | 92 | 83 |

1. A spraying liquid suitable for spraying plants, comprising
a. biomass comprising spores of fusaricidin producing bacteria, in an amount of 50 g per hectare to 2000 g per hectare,
b. at least one category 1 additive or at least one category 2 additive or a mixture of at least one category 1 additive and at least one category 2 additive, in a total amount of 20 g per hectare to 2000 g per hectare, and
c. a total amount of less than 81 g per hectare category 3 additives,
wherein the at least one category 1 additive is selected from at least one group of additives 1a) to 1f):
1a) poloxamers, polyethylene glycol (A)—polypropylene glycol (B) block copolymers of the A-B-A or B-A-B type, comprising polyethylene glycol-polypropylene glycol-polyethylene glycol block polymers with a molar mass of approximately 950 to 6500 g/mol and a polyethylene oxide content of 10% to 50% w/w;
1b) polyvinylpyrolidines, polyvinylacetates, and statistical and block copolymers of polyvinyl pyrrolidone and polyvinyl acetate;
1c) polyoxyethylene sorbitol fatty acid esters (“polysorbates”), having a fatty acid chain with a length of 10 to 20 carbon atoms, from 10 to 150 oxyethylene units;
1d) fatty acid glycerol esters including fatty acid mono- and diesters of glycerol and polyglycerol, glycerolmonooleate, glyceroldioleate;
1e) alcohol alcoxylates;
1f) alkyl glucosides having an alkyl chain length C5-C15 and 1 to 10 glucoside units,
and
wherein the at least one category 2 additive is selected from at least one group of additives 2a) to 2c)
2a) polyethylene glycol-grafted polyacrylates, and salts thereof;
2b) ethoxylated polyethyleneimines;
2c) sodium lauryl ether sulfate,
wherein the category 3 additive belongs to a group of additives 3a) to 3g)
3a) lignosulfonates;
3b) alkylbenzene sulfonates and salts thereof;
3c) alkylnaphthalene sulfonates and salts thereof;
3e) napthalene sulfonic acid co-condensates;
3f) alkyl sulfonates and salts thereof;
3g) fatty alcohol sulfates and salts thereof.

2. The spraying liquid according to claim 1, wherein the at least one category 1 additive of the groups 1a) to 1f) is selected from:
for group 1a): poloxamers;
for group 1 b) statistical copolymers of polyvinyl pyrrolidone and polyvinyl acetate;
for group 1c) polyoxyethylene sorbitol oleic acid esters with 20 to 40 oxyethylene units;
for group 1d) fatty acid mono- and diesters of glycerol and polyglycerol for group 1e) alcohol alcoxylates with 5 to 80 oxyethylene units;
for group 1f) alkyl polyglucosides having an alkyl chain length C8-C10 and mono- and diglucosides with an overall polymerization degree of 1.5.

2. The spraying liquid according to claim 1, wherein the at least one category 1 additive of the groups 1a) to 1f) is selected from:
for group 1a) from polyethylene—polypropylene glycol block polymer with 10% ethyleneoxide, 850 g/mol, polyethylene—polypropylene glycol block polymer with 40% ethyleneoxide, 3250 g/mol, polyethylene—polypropylene glycol block polymer with 20% ethyleneoxide, 3250 g/mol, or polyethylene—polypropylene glycol block polymer with 50% ethyleneoxide, 1750 g/mol;
for group 1 b) polyvinylpyrrolidone with an average molar mass of maximally 50 000 g/mol, or polyvinylpyrrolidone-polyvinylacetate statistical copolymer;
for group 1c) polyoxyethylene (40) sorbitol hexaoleate, sorbitol ethoxylate esters, poly oxyethylene (20) sorbitan monolaurate, poly(oxyethylene) (20) sorbitan monooleate, sorbitan monooleate or sorbitol ethoxylate ester of polymerized fatty acid;
for group 1d) mixtures of polyglycerolesters and fatty acid esters, or glycerolmon/diooleat;
for group 1e) alkyl polyethylene glycol ether from C10-Guerbet alcohol, HLB 13, alkyl polyethylene glycol ether from C10-Guerbet alcohol, HLB 16, polyoxyethylene (23) lauryl ether or alkyl polyethylene glycol ether from C16 C18 fatty alcohol, HLB 18.5;
for group 1f) alkyl polyglucoside.

3. A kit of at least two parts to prepare a spraying liquid according to claim 1, wherein the biomass comprising spores of fusaricidin producing bacteria are provided in a first concentrated form and at least one of the category 1 additive and/or category 2 additive and/or category 3 additive are provided in at least one further concentrated forms and wherein the relative amounts of the biomass comprising spores of fusaricidin producing bacteria and the relative amounts the category 1 additive and/or the category 2 additive and/or the category 3 additive in the kit are adapted to provide the amounts described in any one of the preceding claims.

4. The kit of parts according to claim 4, wherein the spraying liquid is prepared to be used in a method to control phytopathogenic fungi.

5. A method to control phytopathogenic fungi wherein a spraying liquid according to claim 1 is sprayed on plants.

6. The method to control phytopathogenic fungi according to claim 6, wherein the spraying liquid is sprayed in an amount of about 100 liter to about 1000 liter per hectare and the spraying liquid comprises biomass comprising spores of fusaricidin producing bacteria in an amount of 0.005% w/w to 2% w/w and the category 1 additive and/or the category 2 additive in a total amount of 0.005% w/w to 2% w/w.

7. The method to control phytopathogenic fungi according to claim 6, wherein the plants are selected from maize, small-grain cereals, cotton, canola, sunflowers, tobacco, peanuts, vegetables, grapevines, leafy vegetables, root & tuber vegetables, onions, strawberries, blueberries, strawberry, black currant, white currant, red currant, blackberry, raspberry, tomato, pepper, chilli, cucumber, aubergine, melons, watermelons, cucumbers, tomatoes, peppers & aubergines, herbs, turf and ornamentals.

8. The method to control phytopathogenic fungi according to claim 6, wherein the phytopathogenic fungi is Alternaria spp., Botrytis cinerea or Phytophthora infestans.

9. A solid or liquid formulation comprising:
a. 5% w/w to 50% w/w biomass comprising spores of fusaricidin producing bacteria,
b. 5% w/w to 50% w/w of at least one category 1 additive or at least one category 2 additive or a mixture of at least one category 1 additive or at least one category 2 additive, and
c. less than 2% w/w of category 3 additives,
wherein the at least one category 1 additive is selected from at least one group of additives 1a) to 1f):
1a) poloxamers, polyethylene glycol, polypropylene glycol or polyethylene glycol block polymers with a molar mass of approximately 950 to 6500 g/mol and a polyethylene oxide content of 10% to 50% w/w;
1b) polyvinylpyrolidines, polyvinylacetates and statistical and block copolymers of polyvinyl pyrrolidone and polyvinyl acetate, all with a molar mass of approximately 9000 to 70000 g/mol;
1c) polyoxyethylene sorbitol fatty acid esters (“polysorbates”), having a fatty acid chain with a length of 10 to 20 carbon atoms, from 10 to 150 oxyethylene units;
1d) fatty acid glycerol esters, fatty acid mono- and diesters of glycerol and polyglycerol, glycerolmonooleate, glyceroldioleate, polyglycerol fatty acid ester;
1e) alcohol alcoxylates, from 5 to 80 oxyethylene units;
1f) alkyl glucosides having an alkyl chain length C5-C15 and 1 to 10 glucoside units,
and the at least one category 2 additive is selected from at least one group of additives 2a) to 2c)
2a) polyethylene glycol-grafted polyacrylates, and salts thereof;
2b) ethoxylated polyethyleneimines;
2c) sodium lauryl ether sulfate,
and the category 3 additives are selected from the groups of additives 3a) to 3g)
3a) lignosulfonates;
3b) alkylbenzene sulfonates and salts thereof;
3c) alkylnaphthalene sulfonates and salts thereof;
3e) apthalene sulfonic acid co-condensates;
3f) alkyl sulfonates and salts thereof;
3g) fatty alcohol sulfates and salts thereof.

10. The solid or liquid formulation according to claim 10, comprising:
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives,
b. at least one polyethylene—polypropylene glycol block polymer with 50% ethyleneoxide, 1750 g/mol and at least one mixture of polyglycerolesters and fatty acid esters as category 1 additives,
c. at least one polyethylene—polypropylene glycol block polymer with 50% ethyleneoxide, 1750 g/mol and at least one polyethylene—polypropylene glycol block polymer with 40% ethyleneoxide, 3250 g/mol as category 1 additives,
d. at least one poly(oxyethylene) (20) sorbitan monooleate and at least one sorbitan monooleate as category 1 additives, or
e. at least one polyethylene—polypropylene glycol block polymer with 10% ethyleneoxide, 850 g/mol and at least one polyvinylpyrrolidone with an average molar mass of maximally 50 000 g/mol as category 1 additives.

11. The solid formulation according to claim 10, comprising 2% w/w to 50% w/w of a category 1 additive, wherein the category 1 additive is a combination of
a. at least one polyethylene—polypropylene glycol block polymer with 50% ethyleneoxide, 1750 g/mol and at least one polyethylene—polypropylene glycol block polymer with 20% ethyleneoxide, 3250 g/mol as category 1 additives,
b. at least one polyethylene—polypropylene glycol block polymer with 50% ethyleneoxide, 1750 g/mol and at least one mixture of polyglycerolesters and fatty acid esters as category 1 additives,
c. at least one poly(oxyethylene) (20) sorbitan monooleate and at least one sorbitan monooleate, or
d. at least one polyethylene—polypropylene glycol block polymer with 10% ethyleneoxide, 850 g/mol and at least one polyvinylpyrrolidone with an average molar mass of maximally 50 000 g/mol, as category 1 additives.

12. The liquid formulation according to claim 10, comprising:
a. 2% w/w to 15% w/w of at least one category 1 additive selected from polyethylene—polypropylene glycol block polymer with 50% ethyleneoxide, 1750 g/mol, polyethylene—polypropylene glycol block polymer with 20% ethyleneoxide, 3250 g/mol, polyethylene—polypropylene glycol block polymer with 40% ethyleneoxide, 3250 g/mol and/or a mixture of polyglycerolesters and fatty acid esters, or a mixture of at least two of these, and
b. 1% w/w to 15% w/w of at least one category 2 additive selected from methylmethacrylate graft copolymer.

13. A process for making a solid formulation comprising:
a. 5.0% w/w to 50% w/w biomass comprising spores of fusaricidin producing bacteria,
b. 5.0% w/w to 50% w/w of a category 1 additive,
c. 0% w/w to 20% w/w of a category 2 additive,
d. 0% w/w to 2% w/w of a category 3 additive, and
e. 30% w/w to 50% w/w of auxiliaries.
wherein the total amounts of a, b, c, d, and e are selected to result in a total amount of 100% w/w,
comprising the following steps:
A. providing biomass comprising spores of fusaricidin producing bacteria,
B. dispersing said biomass in a continuous phase comprising,
C. adding at least one category 1 additive,
D. optionally adding at least one category 2 additive,
E. optionally adding at least one category 3 additive,
F. optionally adding auxiliaries,
G. drying the biomass containing slurry by spray-drying, vacuum-drum drying, fluidized-bed drying or spray-granulation,
Wherein steps C., D., E. and F. can be carried out at any time during the process.

14. A process for making a suspension concentrate (SC) formulation comprising:
a. providing biomass comprising spores of fusaricidin producing bacteria,
b. dispersing said biomass by shear or mechanical energy input in a continuous phase comprising,
c. adding at least one category 1 additive,
d. optionally adding at least one category 2 additive,
e. optionally adding at least one category 3 additive,
f. optionally adding auxiliaries,
g. wherein steps c), d), e) and f) can be carried out at any time during the process.
wherein the total amounts of a, b, c, d, and e are selected to result in a total amount of 100% w/w,
comprising the following steps:
H. providing biomass comprising spores of fusaricidin producing bacteria,
I. dispersing said biomass in a continuous phase comprising,
J. adding at least one category 1 additive,
K. optionally adding at least one category 2 additive,
L. optionally adding at least one category 3 additive,
M. optionally adding auxiliaries,
N. drying the biomass containing slurry by spray-drying, vacuum-drum drying, fluidized-bed drying or spray-granulation,
Wherein steps C., D., E. and F. can be carried out at any time during the process.

15. A process for making a suspension concentrate (SC) formulation comprising:
a. providing biomass comprising spores of fusaricidin producing bacteria,
b. dispersing said biomass by shear or mechanical energy input in a continuous phase comprising,
c. adding at least one category 1 additive,
d. optionally adding at least one category 2 additive,
e. optionally adding at least one category 3 additive,
f. optionally adding auxiliaries,
g. wherein steps c), d), e) and f) can be carried out at any time during the process.
